(19) United States
(12) Patent Application Publication
(10) Pub. No.: US 2010/0129478 A1
(43) Pub. Date: May 27, 2010

(54) Plant Extracts and Methods and Uses Therefore

(76) Inventors: Villoo Morawala Patell, Bangalore (IN); Vyas Dhruvdev, Bangalore (IN)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 11/816,142

(22) PCT Filed: Feb. 23, 2005
(86) PCT No.: PCT/IB05/00474

§ 371 Date: Aug. 13, 2007

(51) Int. Cl. A61K 36/00; A61P 3/04; A61P 35/00; A61P 3/10; A61P 9/00

(52) U.S. Cl. 424/769

(57) ABSTRACT

The invention relates to compositions such as pharmaceutical compositions and comestibles comprising extracts of Terminalia arjuna and methods for preventing or treating diabetes, obesity, cardiovascular disease, cancer and related disease conditions in mammals, such as humans, and uses therefore.

### FIELD OF INVENTION

The invention relates to extracts from Terminalia plant species that are capable of being used in methods for managing disease such as diabetes, obesity, cardiovascular diseases, and cancer, and related disease conditions. More particularly, the invention relates to certain extracts from Terminalia arjuna, their uses as agents for the treatment of certain diseases such as diabetes, obesity, cardiovascular disease and cancer in mammals, particularly humans, processes for obtaining them and delivery formats therefore.

### BACKGROUND OF THE INVENTION

### Diabetes Mellitus

Diabetes mellitus is a common, serious disease characterized by hyperglycemia. The disease can be divided into two major subclasses: insulin-dependent diabetes mellitus (IDDM), also known as type I diabetes, and non-insulin-dependent diabetes mellitus (NIDDM), also known as type II diabetes (World Health Organization Study Group. Diabetes mellitus. WHO Tech. Rep. Ser. 727:1-113, 1985). IDDM results from insulin deficiency caused by cell-mediated autoimmune destruction of pancreatic beta cells, and generally develops in the young (Yoon J W., Insulin-dependent diabetes mellitus. In: Roitt I M and Delves P J. (Eds.) Encyclopedia of Immunology, Second Edition. Academic Press Ltd., London, pp. 1390-1398, 1998; Bach J F., Insulin-dependent diabetes mellitus as a beta cell targeted disease of immunoregulation. J. Autoimm. 8:439-463, 1995). IDDM accounts for approximately 10-15% of the diabetic population) worldwide (World Health Organization Study Group. Diabetes mellitus. WHO Tech. Rep. Ser. 727:1-113, 1985). In contrast, NIDDM results from a variable combination of insulin resistance and insulin deficiency, and generally develops in adults (Jun H S, et al., Pathogenesis of non-insulin-dependent (Type II) diabetes mellitus (NIDDM)—Genetic predisposition and metabolic abnormalities. Advanced Drug Delivery Reviews 35:157-177, 1999; DeFronzo R A., The triumvirate: .beta.-cell, muscle, liver: a collusion responsible for NIDDM. Diabetes 37:667-687, 1988). However, NIDDM can also develop at a younger age, as seen in the maturity-onset diabetes of the young (Pirart J., Diabetes mellitus and its degenerative complications: a prospective study of 4400 patients observed between 1947 and 1973. Diabetes Care 1:168-188, 1978). NIDDM accounts for over 85% of the diabetic population worldwide. Both IDDM and NIDDM can cause microvascular and macrovascular complications, resulting in increases in morbidity and mortality (Fajans S S, et al., Prediabetes, subclinical diabetes, and latent clinical diabetes: interpretation, diagnosis and treatment. In: Leibel D S, Wrenshall G S. (Eds.) On the Nature and Treatment of Diabetes. Excerpta Medica, Amsterdam, pp. 641-656, 1965).

NIDDM is a complex disease that is currently thought to be influenced by more than a single gene or environmental factor (Ghosh S, et al., Genetic analysis of NIDDM. Diabetes 45:1-14, 1995; Kobberling J. Studies on the genetic heterogeneity of diabetes mellitus. Diabetologia 7:46-49, 1971; Rotter J L, et al., Genetics of diabetes mellitus. In: Rifkin H, Porte D (Eds.) Diabetes Mellitus Theory and Practice. Elsevier, N.Y., pp. 378-413, 1990). Familial aggregation and the high concordance rate for the disease (60-100%) in identical twins suggest that genetic factors play an important role in the pathogenesis of NIDDM (O'Rahilly S, et al., Type 2 (noninsulin dependent) diabetes mellitus. New genetics for old nightmares. Diabetologia 31:407-414, 1988; Barnett A H, et al., Diabetes in identical twins. A study of 200 pairs. Diabetologia 20:87-93, 1981). In addition, environmental factors such as obesity, physical activity and diet also play a strong role in the development of the disease (Knowler W C, et al., Gm and type 2 diabetes mellitus: an association in American Indians with genetic admixture. Am. J. Hum. Genet. 43:520-526, 1988; Bennett P H, et al., Epidemiology and natural history of NIDDM: non-obese and obese. In: Alberti KGMM, DeFronzo R A, Keen H, Zimmett P (Eds.) International Textbook of Diabetes Mellitus. Wiley, N.Y., pp. 147-176, 1992; Helmrich S P, et al., Physical activity and reduced occurrence of NIDDM. N. Engl. J. Med 325:147-152, 1991). Although the relative contribution of genetic and environmental factors to the development of NIDDM differs among individuals, patients generally have two common metabolic abnormalities, insulin resistance and defects in glucose-stimulated insulin secretion, which lead to the disease state (Saad M F, et al., A two step model for development of non-insulin-dependent diabetes. Am. J. Med. 90:229-235, 1991; DeFronzo R A, et al., Pathogenesis of NIDDM: A balanced overview. Diabetes Care 15:318-368, 1992; Lillioja S, et al., Insulin resistance and insulin secretory dysfunction as precursors of non-insulin-dependent diabetes mellitus. N. Engl. J. Med. 329:1988-1992, 1993).

The insensitivity of the target tissue in response to insulin (insulin resistance) appears to develop first in genetically predisposed subjects in the presence of the necessary environmental factors (Jun H S, et al., Pathogenesis of non-insulin-dependent (Type II) diabetes mellitus (NIDDM)—Genetic predisposition and metabolic abnormalities. Advanced Drug Delivery Reviews 35:157-177, 1999). To compensate for this, that is, to lower blood glucose and maintain normoglycemia, the secretion of insulin from the beta cells increases, resulting in hyperinsulinemia. Over time, the insulin resistance worsens, and the compensatory action fails, leading eventually to impaired glucose tolerance. Insulin secretion reaches a plateau, and beta cell function is impaired, resulting in insulin deficiency, and leading finally to hyperglycemic NIDDM. In addition, hyperglycemia itself leads to impaired insulin resistance and insulin secretion, exacerbating the disease.

The regulation of diet and exercise and/or treatment with insulin or hypoglycemia drugs has been used for the control of diabetes. Treatment with these agents is successful in some cases, but the mortality index continues to rise. Insulin treatment provides symptomatic relief rather than a cure for NIDDM. Hypoglycemic agents such as sulfonylureas and biguanides (metformin) also lower blood glucose, but again, simply provide symptomatic relief. Sulfonylureas lower the blood glucose level by stimulating the release of insulin from pancreatic beta cells. These agents directly stimulate insulin release by closing adenosyl triphosphate (ATP)-sensitive potassium channels and depolarizing the cell membrane (Aguilar-Bryan L, et al., Cloning of the beta cell high-affinity sulfonylurea receptor: a regulator of insulin secretion. Science 268:423-426, 1995; Tan G H, et al., Pharmacologic treatment options for non-insulin-dependent diabetes mellitus. Mayo Clinic Proceedings 71:763-768, 1996; Lubbos H, et al., Oral hypoglycemic agents in type II diabetes mellitus. American Family Physician. 52:2075-2078, 1995) The side effects of sulfonylureas include hypoglycemia, renal and hepatic disease, gastrointestinal disturbances, increased cardiovascular mortality, dermatological reactions, dizziness, drowsiness and headache. Biguanides lower blood glucose levels by reducing intestinal glucose absorption and hepatic glucose, but not by stimulating insulin secretion. The major side effects of biguanidine are lactic acidosis and increased cardiovascular mortality. Alpha glucosidase inhibitors inhibit intestinal alpha glucosidases and consequently delay the digestion of sucrose and complex carbohydrates. The side effects of alpha glucosidase inhibitors include gastrointestinal side effects and hypoglycemia. Thiazolidinediones improve insulin resistance directly, enhancing the effects of circulating insulin, directly stimulate peripheral glucose uptake and inhibit glucose production in the liver. Thiazolidinediones are only effective in the presence of insulin and may cause red blood cell abnormalities, and headache.

Therefore, more effective drugs for the treatment of diabetes are clearly needed.

### Obesity

It is generally recognized in the medical profession that obesity among the populations of the United States and some other countries has been steadily increasing in the last several decades.

Obesity is a condition characterized by excessive accumulation of fat on the body. Obesity can be measured by either body weight or by body mass index (BMI). By convention, obesity is said to be present when body weight exceeds by 20 percent the weight listed in typical height-weight index tables. The other measurement of obesity, BMI, is the amount of fat present in the body and is considered a reliable indication of fatness in non-athletic adults. The BMI may be calculated by using the following formula: BMI equals [body weight in kg] divided by [height in meters].sup.2. In general, a normal BMI is between the range of 20 to 25, whereas the BMI of obese individuals is greater than or equal to 30.

Such obesity has caused or contributed to marked increases in the occurrence of heart disease, hypertension, diabetes, osteoarthritis of the knees and hips, and increased morbidity resulting from related medical conditions. It has been reported that 50% of all American adults are overweight.

There are a variety of methods commonly used for treating obesity. These include dietary adjustments such as caloric restriction, exercise regimens, pharmacotherapy, surgery, and psychotherapy. However, there are problems associated with many of these treatments.

The use of human growth (hGH) hormone to treat obesity is described in U.S. Pat. No. 5,597,595. hGH, the most abundant hormone secreted by the pituitary gland, is the primary hormone responsible for growth in mammals, accelerating lipolysis (fat metabolism), and protein synthesis. hGH secretion is markedly decreased in obese individuals. hGH interacts with specific growth hormone receptors, which are widely distributed throughout the body. For example, fat cells (adipocytes) have hGH receptors, and when hGH binds to these receptors, it stimulates the adipocytes to breakdown triglycerides as well as suppresses their ability to take up and accumulate circulating lipids. Administration of hGH needs to be closely monitored, however, as diabetes mellitus may develop in individuals receiving hGH treatment. Diabetes may result because hGH reduces the amount of glucose uptake by cells, thus causing an increase of blood glucose concentration. Not only does this increase in blood glucose levels stimulate the .beta.-cells of the pancreas to secrete extra insulin, but hGH also directly stimulates the .beta.-cells to produce insulin. The combination of these two stimulatory effects so greatly overstimulates insulin secretion that the .beta.-cells literally “burn out.” When this occurs, diabetes mellitus may develop. Guyton and Hall; TEXTBOOK OF MEDICAL PHYSIOLOGY, 938 (1995).

One of the best ways to induce weight loss is through dieting or caloric restriction, i.e., establish a caloric deficit by bringing intake below output. The most common method of reducing caloric intake is by means of a low calorie diet. There are, however, a couple of common pitfalls to dieting. One is that when the dieter stops the diet and returns to the usual fare, the incentives to overeat are multiplied. Second, many obese people choose novel or bizarre diets. One example of this type of diet is a high-fat/high-protein diet. These diets are known as ketogenic diets because they contain a high level of cholesterol, which facilitates ketosis. Ketosis is associated with nausea, hypotension, and lethargy. Kaplan et al., supra, 735.

Pharmacotherapy may include the administration of drugs that function by increasing serontonin levels in the brain and ultimately controlling appetite. Serotonin (5-hydroxytryptamine, 5HT) is related to several general behaviors such as sleep, emotion, sex, and appetite. The drug Redux™ (dexfenfluramine), which is commonly used as a weight control medication, functions by increasing serotonin levels. Specifically, Redux™ functions by inhibiting re-uptake of serotonin and at the same time stimulating release of serotonin. Several controlled clinical trials have demonstrated that Redux™ can help individuals maintain caloric restriction and a lower body weight for a least a year. It should also be noted that Redux™ appears to reduce appetite in obese more than non-obese individuals. Katzung, BASIC & CLINICAL PHARMACOLOGY 276 (1998). However, there are some adverse side-effects affiliated with Redux™ such as headache, insomnia, and pulmonary hypertension. Id.

Surgical methods that cause malabsorption of food or reduce gastric volume have been widely used in people who are markedly obese. Gastric bypass is a procedure used to make the stomach smaller. In particular, the stomach is reduced by stapling or transecting one of the curvatures of the stomach. The primary objective of reducing the volume of the stomach is to slow the passage of food, thereby limiting the amount of food the individual can intake. Although the results are successful, many adverse side effects result. For example, vomiting, electrolyte imbalance, and obstructions may occur. An alternate surgical procedure is lipectomy, which is the removal of fat. This technique is primarily used for cosmetic purposes, however, and has no real impact on weight loss in the long run. Kaplan et al., supra, 276.

Hence, for all of the reasons detailed above, a better method for facilitating weight loss in obese or overweight individuals that is readily available and cost-efficient is needed.

### Cardiovascular Diseases

Cardiovascular disease (CVD) is a leading cause of mortality and is responsible for one-third of all global deaths. Nearly 85% of the global mortality and disease burden from CVD is borne by low- and middle-income countries. In India, for example, approximately 53% of CVD deaths are in people younger than 70 years of age; in China, the corresponding figure is 35%. The majority of the estimated 32 million heart attacks and strokes that occur every year are caused by one or more cardiovascular risk factors—hypertension, diabetes, smoking, high levels of blood lipids, and physical inactivity—and most of these CVD events are preventable if meaningful action is taken against these risk factors.

A staggering number of people are destined to experience a cardiovascular-related disorder sometime in their lives. Almost 1 million Americans die each year as a result of cardiovascular disease, whereas 556,000 die each year from cancer. Despite these facts, people are often more afraid of cancer than they are of vascular disease.

Americans have become complacent about the dangers of arterial disease. One reason is that the percentage of young people dying from acute heart attack has plummeted over the past 50 years. Explanations for these reductions include lifestyle changes, greater use of dietary supplements/preventive medications, and improved cardiac medical care.

The question is why are so many Americans continuing to die from heart attack and stroke? The fundamental answer is that people are living longer. What has happened is that much of the human population has succeeded in delaying the development of arterial disease. So instead of suddenly dying from a heart attack at age 50, the vascular symptoms do not manifest until the 60s or 80s are reached. At this point, systemic arteriosclerosis has damaged the major organ systems, and multiple degenerative diseases result in diminished quality and quantity of life.

Many of the underlying causes of arterial disease have been identified in the scientific literature. Regrettably, cardiologists have only addressed a limited number of these factors, such as prescribing cholesterol-lowering drugs, controlling hypertension, etc. By ignoring the other proven causes for the epidemic of vascular-related diseases, a significant number of Americans are experiencing needless suffering and are dying prematurely.

### Terminalia Arjuna Plant Extracts

Terminalia arjuna is a deciduous tree found throughout India growing to a height of around 60-90 feet. Terminalia arjuna belongs to the family Combretaceae and is called “Arjuna” in vernacular. Terminalia arjuna has been used for over 1500 years in India as a cardio tonic and has been indicated for derangement of all three humoursin, vata, pitta and kapha in Ayurveda. The bark of Terminalia arjuna has been widely used in Indian system of medicine for a variety of purposes.

U.S. Pat. No. 6,162,438 describes an edible herbal compositions, mixture of at least three, preferably at least six, herbs selected from the group consisting of Terminalia arjuna, Cynara scolymus, Zingibar officinale, Allium sativum, Crataegus oxycantha, Curcuma longa, Boerhaavia diffusa and Trigonella foenumgraecum, for use as agents for the control of hypertension, hyper-cholesterolemia and hyperlipidemia in mammals.

Sharma V N et al. evaluated the antioxidant and hypocholesterolaemic effects of Terminalia arjuna tree bark (a popular cardiotonic substance in Indian pharmacopoeia) and compared it with a known antioxidant, vitamin E by a randomised controlled trial. It was concluded from this trial that, Terminalia arjuna tree bark powder has significant antioxidant action that is comparable to vitamin E. In addition, it also has a significant hypocholesterolaemic effect. (Antioxidant and hypocholesterolaemic effects of Terminalia arjuna tree-bark powder: a randomised placebo-controlled trial, J Assoc Physicians India 2001 February; 49:231-235)

Bioassay-guided separation methods was used to determine the cancer cell growth inhibitory constituents residing in the bark, stem and leaves of the Mauritius medicinal plant Terminalia arjuna (Combretaceae) by Chapuis et. al. The cancer cell line active components were found to be gallic acid, ethyl gallate, and the flavone luteolin. Only gallic acid was previously known to occur in this plant. Luteolin has a well-established record of inhibiting various cancer cell lines and may account for most of the rationale underlying the use of T. arjuna in traditional cancer treatments. Luteolin was also found to exhibit specific activity against the pathogenic bacterium Neisseria gonorrhoeae. (Antineoplastic agents 338. The cancer cell growth inhibitory constituents of Terminalia arjuna (Combretaceae). J Ethnopharmacol. 1996 August; 53(2):57-63).

The bark of Terminalia arjuna tree has a long history of use as a cardiac tonic as well, and has been indicated in the treatment of coronary artery disease, heart failure, hypercholesterolemia and for relief of anginal pain. (Miller, A. L. Botanical Influences on cardiovascular disease. Alternative Medicine Review. December 1998, vol 3. No. 6, pages 421-431.

Water and methanol extracts of stem bark of Terminalia arjuna were found to inhibit the HIV-1 activity by more than 70% at 0.2 mg/ml. (Kandil F. E. et al. A Tannin anti-cancer promoter from Terminalia arjuna. April 1998, Vol 47. No. 8, pages 1567-1568.

Adjuvant therapy with Terminalia arjuna bark powder in post Myocardial Infarction angina patients produced relief in angina alongwith improvement in left ventricular ejection fraction (LVEF) and left ventricular mass (LVM). (Dwivedi, S. et al. Beneficial effects of Terminalia arjuna in coronary Artery Disease. Indian Heart Journal. September-October 1997, vol. 49. No. 5, pages 507-510.)

Ethanolic extract of Terminalia arjuna tree bark in doses of 100 mg/kg and 500 mg/kg significantly reduced total and LDL cholesterol levels in hypercholesterolaemic rabbits. (Ram et al. Hypocholesterolaemic effects of Terminalia arjuna tree bark. Journal of Ethnopharmacology. Vol 55. No. 3, pages 165-169.)

There is no data or technical information in the prior art, which indicates the use of the Terminalia arjuna extracts in the treatment or prevention or management of Diabetes and Obesity.

It is reported that water and methanol extracts of stem of T.-arjuna has shown to have HIV-1 activity. But there are no other references to the use of T. arjuna bark extract with successive extraction solvent system, in the treatment of Cancer.

Terminalia arjuna plant has been known for its cardiotonic effect. Also there are reports indicating the use of T. arjuna in the treatment of coronary artery disease, heart failure, hypercholesterolemia and for relief of anginal pain. But there are no clear cut evaluation of the mechanisms of action responsible for these activities. In the present invention however, we have established that cells treated with Terminalia arjuna extracts showed marked decrease in the amount of total lipids. Also the said extracts have shown 3-Hydroxy-3-methylglutaryl-coenzyme A (HMG-CoA) reductase inhibition activity. HMG-CoA is the first committed step in cholesterol biosynthetic pathway. HMG-CoA reductase the rate-limiting enzyme step in cholesterol biosynthesis has become the most successful drug treatment for lowering total plasma cholesterol, in particular LDL-cholesterol.

There exists a need for the development of new medicines which are effective in treating obesity, cardiovascular disease, certain cancers, and diabetes and related diseases.

### SUMMARY OF THE INVENTION

Objects of the invention will become apparent from the following description and examples.

The present invention relates to certain extracts of plant parts from plants of the Terminalia species, such as Terminalia arjuna that are indicated as being potentially useful in the treatment of diseases such as obesity, cardiovascular disease, certain cancers, diabetes and related diseases. Furthermore, the invention relates to prophylactic compositions comprising extracts of Terminalia species, such as extracts from Terminalia arjuna species.

### Brief Description of the Extract Nomenclature:

### Nomenclature of Plant Extracts.

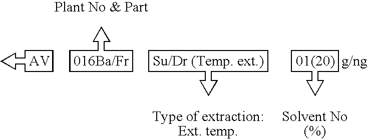 [0036] 1. AV-first two letters represents Avesthagen.
2. Plant Name: The Plants used and in use are assigned with unique 3-digit number, 016 represents Terminalia arjuna.
3. Part of the plant/Tissue: There is a two letter ID for each plant part used. For example Ba for Bark, Fr for whole Fruit.
4. Solvents: The solvents used for extraction are also assigned with two digit numbers 01 for Acetone, 02 for Benzene, 03 for Chloroform, 04 for Ethanol, 05 for Hexane, 06 for Methanol, 07 for Petroleum ether, 08 for water, 09 for ethyl acetate. Percentage of solvents used for extraction is given within bracket (20) for 20% of that solvent. For example if 20% of Ethanol was used for extraction, 04(20).
5. Method of Extraction: Successive extraction is referred to as Su whereas direct extraction is referred to as Di, temperature for extraction is written in bracket. For example, Su(65) represents successive extraction at 65° C.
6. Type of extract, g: gluey and ng: non-gluey.

### BRIEF DESCRIPTION OF THE TABLES AND FIGURES

Table 1: HPLC fingerprint of the extract AV016BaDi(65)04(100).

Table 2: HPLC fingerprint of the extract AV016BaDi(28)04(20).

Table 3: HPLC fingerprint of the extract AV016BaSu(65)09(100).

Table 4: HPLC fingerprint of the extract AV016BaSu(65)01(100).

Table 5: HPLC fingerprint of the extract AV016BaSu(65)01(100)ng.

Table 6: HPLC fingerprint of the extract AV016BaSu(65)01(100)g.

Table 7: HPLC fingerprint of the extract AV016BaSu(65)04(100).

Table 8: HPLC fingerprint of the extract AV016BaSu(65)06(100).

Table 9: HPLC fingerprint of the extract AV016BaSu(105)08(100).

Table 10: HPLC fingerprint of the extract AV016Fr(105)08(100).

Table 11: LC/MS Fingerprint of extract AV016BaDi(28)04(20) (TIC Spectrum (Q1+ve mode)

Table 12: LC/MS Fingerprint of extract AV016BaDi(28)04(20) (TIC Spectrum (Q1 ve mode)

Table 13: LC/MS Fingerprint of extract AV016BaDi(65)04(100) (TIC Spectrum (Q1+ve mode)

Table 14: LC/MS Fingerprint of extract AV016BaDi(65)04(100) (TIC Spectrum (Q1−ve mode)

Table 15: LC/MS Fingerprint of extract AVO16BaSu(65)09(100) (TIC Spectrum (Q1+ve mode)

Table 16: LC/MS Fingerprint of extract AV016BaSu(65)01(100) (TIC Spectrum (Q1+ve mode)

Table 17: LC/MS Fingerprint of extract AV016BaSu(65)01(100) (TIC Spectrum (Q1−ve mode)

Table 18. LC/MS Fingerprint of extract AV016BaSu(65)01(100)ng (TIC Spectrum (Q1+ve mode)

Table 19. LC/MS Fingerprint of extract AV016BaSu(65)01(100)g (TIC Spectrum (Q1+ve mode)

Table 20. LC/MS Fingerprint of extract AV016BaSu(65)04(100) (TIC Spectrum (Q1+ve mode)

Table 21. LC/MS Fingerprint of extract AV016BaSu(65)06(100) (TIC Spectrum (Q1+ve mode)

Table 22. LC/MS Fingerprint of extract AV016BaSu(105)08(100) (TIC Spectrum (Q1+ve mode)

Table 23. LC/MS Fingerprint of extract AV016FrDi(65)04(100) (TIC Spectrum (Q1+ve mode)

Table 24. LC/MS Fingerprint of extract AV016FrSu(105)08(100) (TIC Spectrum (Q1+ve mode)

Table 25. IC₅₀ values of alpha-glucosidase inhibition activity of extracts from different T. arjuna plant parts

Table 26: The effect of insulin and T. arjuna extracts on glucose up-take in 3T3-L1 adipocytes.

Table 27: Anti-obesity potential of Terminalia arjuna extracts.

Table 28: Effect of Terminalia arjuna extracts on lipid levels in CHO-K1 cells.

Table 29: Effect of 20% ethanol extract from T. arjuna bark AV016BaDi(28)04(20) on HMG-CoA reductase activity.

Table 30: Effect of direct 100% ethanol extract from T arjuna bark AV016BaDi(65)04(100) on HMG-CoA reductase activity.

Table 31: Effect of Terminalia arjuna extracts on cell viability of HepG2 cells.

FIG. 1: alpha-Glucosidase inhibition potential of T. arjuna direct ethanol extract, AV016BaDi(65)04(100) and AV016BaDi(28)04(20).

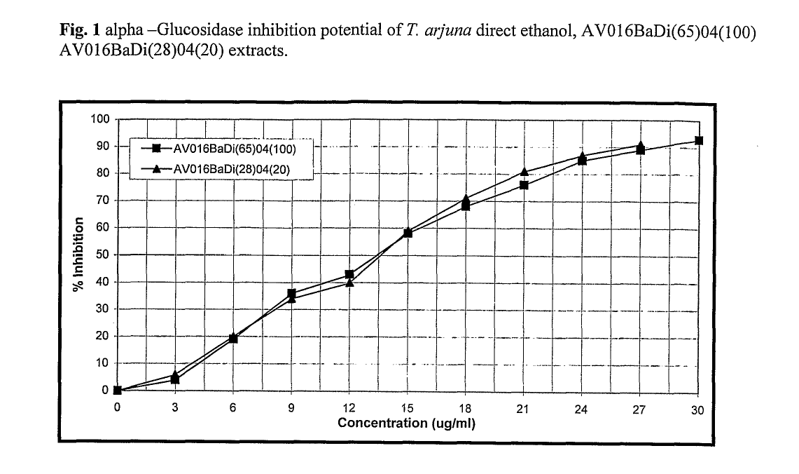

FIG. 2: alpha-Glucosidase inhibition potential of T. arjuna fruit extracts, AV016FrDi(65)04(100) AV016FrSu(65)08(100).

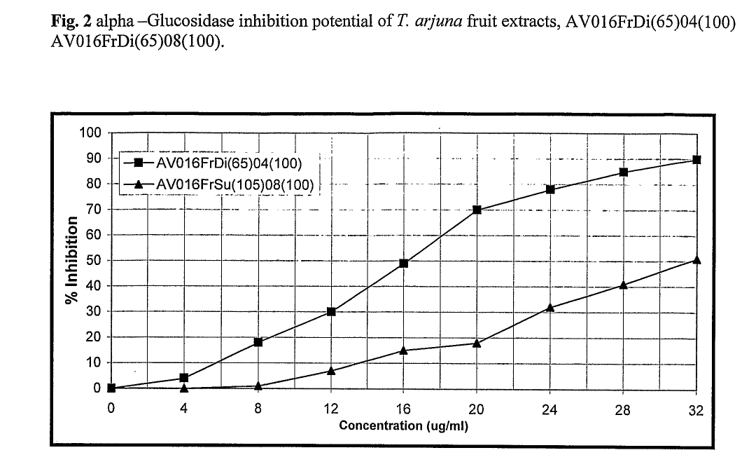

FIG. 3: alpha-Glucosidase inhibition potential of T. arjuna successive extracts, AV016BaSu(65)01(100)g, AV016BaSu(65)01(100)ng, AV016BaSu(65)04(100)

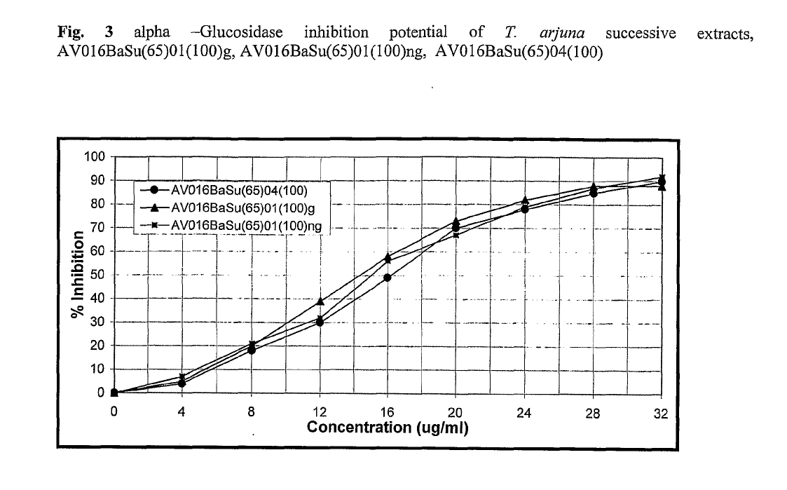

FIG. 4: alpha-Glucosidase inhibition potential of T. arjuna successive methanol and water, AV016BaSu(65)06(100) and AV016BaSu(105)08(100) extracts

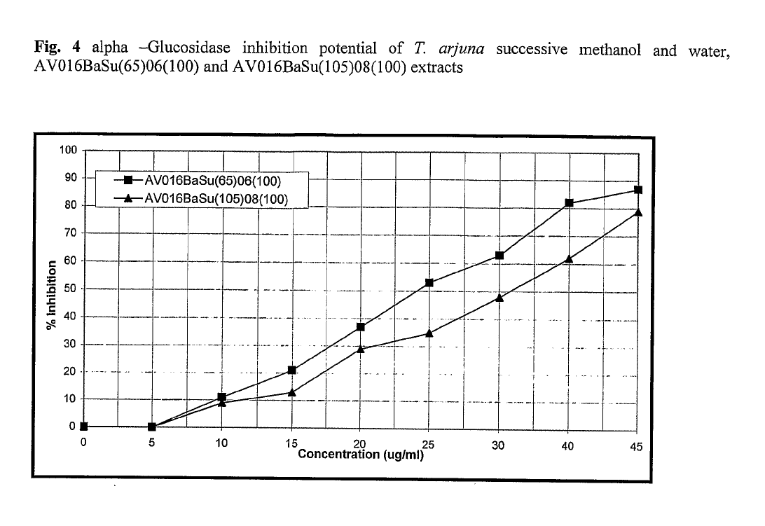

FIG. 5: alpha-Glucosidase inhibition potential of T. arjuna successive AV016BaSu(65)09(100) extracts

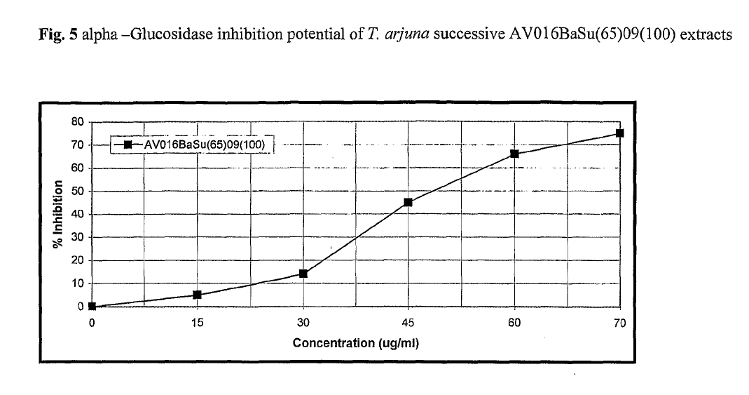

### DETAILED DESCRIPTION OF THE INVENTION

In a first aspect of the invention there is provided a method for treating a disease in a mammal, which comprises administering to the said mammal an effective non-toxic amount of at least an extract from Terminalia arjuna as defined herein. Preferably the mammal is a human being. The skilled addressee will appreciate that “treating a disease” in a mammal means treating, that is to say, alleviating symptoms of the disease and may also mean managing a disease in the sense of preventing such a disease state either advancing ie getting worse or becoming more invasive, or slowing down the rate of advance of a disease.

In a second aspect of the invention, there is a provided a prophylactic method for preventing the occurrence of a disease state in a mammal which comprises administering to the said mammal an effective non-toxic amount of an extract from Terminalia arjuna as defined herein in the preparation of a comestible (=foodstuff) for prophylaxis against the occurrence of a disease state selected from the group diabetes, obesity, cardiovascular disease, and cancer. Preferably the mammal is human and the said extract comprises a single extract from a plant part of Terminalia arjuna or a combination of extracts therefrom as detailed herein. Thus the present invention further relates to extracts which may be isolated from different parts of the Terminalia arjuna plant such as the bark and fruit thereof, the preparation of such extracts, medicaments comprising such extracts, and the use of these extracts and constituents for the preparation of a medicament.

Extracts of the present invention can be isolated from Terminalia tree species, such as Terminalia arjuna, using conventional organic solvent extraction and supercritical fluid extraction technology. Generally, extracts of the invention capable of functioning in a prophylactic or therapeutic manner as outlined herein can be extracted from any Terminalia arjuna plant tissue, such as bark or fruit, depending on the end purpose that is required of the extract.

In a third aspect of the present invention there is provided a process for preparing extracts of the invention from plant parts of Terminalia arjuna that comprises:
(i) Pulverizing selected plant material to a powder;
(ii) Subjecting the powdered plant material to solvent extraction;
(iii) Lyophilizing the obtained extracts.

The choice of selected plant material may be of any type but is preferably selected from the bark or the fruit of the Terminalia arjuna plant.

The solvent extraction process may be selected from direct or successive extraction types such as extraction from plant parts in soxhlet apparatus or in flasks at room temperature or at higher temperature with polar and/or non-polar solvent(s). Typically, the extraction process is as outlined herein.

It will be apparent to the skilled addressee that the selection of solvent, or mixtures of solvents for each step in the isolation of extracts of the invention showing activity can be guided by results of bioassay analysis of separate fractions, for example as indicated herein and/or as shown in the examples.

Also encompassed within the ambit of the invention is a pharmaceutical formulation suitable for use in the treatment of a disease selected from the group diabetes, obesity, cardiovascular disease, and cancer comprising at least one extract as isolated from a Terminalia species, such as Terminalia arjuna, in admixture with a pharmaceutically acceptable carrier. Preferably, the at least one extract is selected from those listed in Tables 1-24 inclusive, depending on design and disease of interest. Preferably the at least one extract is selected from the group of extracts as defined in Tables 25-30 inclusive, again depending on end purpose. Naturally, the skilled addressee will appreciate that such compositions may comprise of two or more plant extracts of the invention in any concentration, which is capable of giving rise to a therapeutic effect. Thus, therapeutic compositions can comprise plant extracts of Terminalia substantially devoid of undesirable contaminating compounds. The plant extracts may have, for example, undergone a number of solvent extraction steps substantially to separate out undesirable components from desirable components such as those alluded to in the examples and aforementioned tables.

The invention thus further provides a method for the treatment of a disease selected from the group obesity, cancer, cardiovascular disease, and diabetes in mammals, including humans, which comprises the use of a clinically useful amount of an extract selected from those listed in Tables 1-24 inclusive, preferably those listed in Tables 25-30 inclusive, in a pharmaceutically useful form, once or several times a day or in any other appropriate schedule for example, orally, or intravenously or by delivery to the lungs in a dry or “wet” spray.

The amount of compound of extract required to be effective in the treatment of the aforementioned diseases will, of course, vary with the disease being treated and is ultimately at the discretion of the medical or veterinary practitioner. The factors to be considered include the condition being treated, the route of administration, and nature of the formulation, the mammal's body weight, surface area, age and general condition and the particular compound to be administered. A suitable effective dose of an extract of the invention generally lies in the range of about 0.01 to about 120 mg/kg bodyweight, e.g. 0.1 to about 120 mg/kg body weight, preferably in the range of about 0.1 to 50 mg/kg, for example 0.5 to 50 mg/kg. The total daily dose may be given as a single dose, multiple doses, e.g. two to six times applications per day. For example, for a 75 kg mammal (e.g. a human) the dose range would be about 8 to 9000 mg per day, and a typical dose could be about 50 mg per day. If discrete multiple doses are indicated treatment might typically be 15 mg of a compound of Formula (I) given up to 4 times per day.

Whilst it is possible for the active extract to be administered alone, it is preferred to present the active extract in a pharmaceutical formulation. Formulations of the present invention, for medical use, comprise an extract of the invention together with one or more pharmaceutically acceptable carriers and optionally other therapeutic ingredients. The carrier(s) should be pharmaceutically acceptable in the sense of being compatible with the other ingredients of the formulation and substantially non-deleterious to the recipient thereof.

The present invention, therefore, further provides a pharmaceutical formulation comprising at least one extract selected from those listed in tables 1-24 inclusive, preferably from those mentioned in tables 25-30 inclusive together with a pharmaceutically acceptable carrier therefore. In a preferment the pharmaceutical formulation comprises at least an extract selected from those listed in tables 25-30, depending on the disease type being treated. Naturally, the skilled addressee will appreciate that when selecting more than one extract from those given in the aforementioned tables for the treatment of any single disease type, that an appropriate selection of extracts fro the disease type will be made. Thus, for example, for the treatment of diabetes, extracts appropriate for doing so will be selected from the said tables.

Naturally, the skilled addressee will appreciate that any pharmaceutical formulation comprising an active extract of the invention can include at least one active extract purified from an extract derived from a Terminalia species. Thus a pharmaceutical formulation may contain more than one active extract derived from two or more Terminalia species.

There is also provided a method for the preparation of a pharmaceutical formulation comprising bringing into association an extract of the invention, and a pharmaceutically acceptable carrier therefore.

Formulations according to the present invention include those suitable for oral or intravenous administration.

Intravenous formulations including at least one extract of the invention and may also be administered in the form of suitable liposomal or niosomal preparations or other suitable delivery vehicle.

Emulgents and emulsion stabilizers suitable for use in the formulation of the present invention include Tween 60, Span 80, cetostearyl alcohol, myristyl alcohol, glycerol mono-stearate and sodium laury sulphate.

The formulations may conveniently be presented in unit dosage form and may be prepared by any of the methods well known in the art of pharmacy. All methods include the step of bringing the active extracts(s) into association with a carrier which constitutes one or more accessory ingredients. In general, the formulations are prepared by uniformly and intimately bringing the active extract(s) into association with a liquid carrier or a finely divided solid carrier or both and then, if necessary, shaping the product into desired formulations.

Formulations of the present invention suitable for oral administration may be presented as discrete units such as capsules, sachets, tablets, lozenges, comprising the active ingredient in a flavoured based, usually sucrose and acacia or tragacanth; pastilles comprising the active ingredient in an inert base such as gelatin and glycerin, or sucrose and acacia; and mouth-washes comprising the active ingredient in a suitable liquid carrier. Each formulation generally contains a predetermined amount of the active extract; as a powder or granules; or a solution or suspension in an aqueous or non-aqueous liquid such as a syrup, an elixir, an emulsion or draught and the like.

A tablet may be made by compression or moulding optionally with one or more accessory ingredients. Compressed tablets may be prepared by compressing an a suitable machine the active extract in a free-flowing form such as a powder or granules, optionally mixed with a binder, (e.g. povidone, gelatin, hydroxypropylmethyl cellulose), lubricant, inert diluent, preservative, disintegrant (e.g. sodium starch glycolate, cross-linked povidone, cross-linked sodium carboxymethyl cellulose), surface active or dispersing agent. Moulded tablets may be made by moulding in a suitable machine a mixture of the powdered extract moistened with an inert liquid diluent. The tablets may optionally be coated or scored and may be formulated so as to provide slow or controlled release of the active ingredient therein using, for example, hydroxypropylmethylcellulose in varying proportions to provide the desired release profile.

A syrup may be made by adding the active extract to a concentrated, aqueous solution of a sugar, for example sucrose, to which may also be added any necessary ingredients. Such accessory ingredient(s) may include flavourings, an agent to retard crystallisation of the sugar or an agent to increase the solubility of any other ingredients, such as a polyhydric alcohol for example glycerol or sorbitol.

In addition to the aforementioned ingredients, the formulations of this invention may further include on or more accessory ingredients) selected from diluents, buffers, flavouring agents, binders, surface active agents, thickeners, lubricants, preservatives (including antioxidants) and the like.

Alternatively, the compositions are dietary supplements, food compositions or beverage compositions suitable for human or animal consumption.

The invention describes the HPLC profiles and Mass spectrums of direct and successive solvent extracts of Terminalia arjuna plant parts thereby giving each extract an identity of itself. The various solvents used for successive extraction are in order from non-polar to polar side i.e hexane, petroleum ether, ethyl acetate, acetone, ethanol, methanol and water. In case of direct extraction alcoholic solvent alone and in combination with water was used as solvent for extraction.

The invention further encompasses novel extracts defined by reference to their HPLC and MS fingerprints as defined in Tables 1-24 inclusive, which are isolated from different parts of Terminalia arjuna plant, the preparation of such extracts, the medicaments containing said extracts, and the use of these extracts and constituents for the preparation of a medicament.

In a further aspect of the invention, the pharmaceutical formulations for preventing, treating, or managing diabetes and diabetes related disorders, comprise direct extracts of T. arjuna bark with 100% ethanol solvent [AV016BaDi(65)04(100)] and 20% ethanol solvent [AV016BaDi(28)04(20)], successive extracts of T. arjuna bark with ethyl acetate solvent [AV016BaSu(65)09(100)], successive extract of T arjuna bark with acetone solvent [AV016BaSu(65)01(100)g], successive extracts of T. arjuna bark with acetone solvent [AV016BaSu(65)01(100)ng], successive extracts of T. arjuna bark with ethanol solvent [AV016BaSu(65)04(100)], successive extracts of T. arjuna bark with methanol solvent [AV016BaSu(65)06(100)] and successive extracts of T. arjuna bark with water solvent [AV016BaSu(105)08(100)], direct extract of T. arjuna fruit with ethanol solvent [AV016FrDi(65)04(100)] and successive extract of T. arjuna fruit with water solvent [AV016FrSu(105)08(100)], alone or in combination thereof. Such pharmaceutical formulations may contain a pharmaceutically acceptable carrier, excipient, or diluent as outlined herein.

In a further aspect of the invention, the pharmaceutical formulations for preventing, treating, or managing obesity and obesity related disorders, comprise direct extracts of T. arjuna bark with 100% ethanol solvent [AV016BaDi(65)04(100)] and 20% ethanol solvent [AV016BaDi(28)04(20)], successive extracts of T. arjuna bark with bark ethyl acetate, gluey acetone, non-gluey acetone, ethanol and methanol extracts AV016BaSu(65)09(100), AV 016BaSu(65)01(100)_{g}, AV016BaSu(65)01(100)ng, AV016BaSu(65)04(100), AV016BaSu(65)06(100) and AV016FrDi(65)04(100), alone or in combination thereof. The pharmaceutical formulations may contain a pharmaceutically acceptable carrier, excipient, or diluent as outlined herein.

In a further aspect of the invention, the pharmaceutical formulations for preventing, treating, or managing cardiovascular diseases (CVD) and CVD related disorders, comprise a direct extract of T. arjuna bark with 100% ethanol solvent [AV016BaDi(65)04(100)] and 20% ethanol solvent [AV016BaDi(28)04(20)], successive extract of T. arjuna bark with ethyl acetate [AV016BaSu(65)09(100)] and acetone [AV016BaSu(65)01(100)], alone or in combination thereof. The pharmaceutical formulations may contain a pharmaceutically acceptable carrier, excipient, or diluent as outlined herein.

In a further aspect of the invention, the pharmaceutical formulations for preventing, treating, or managing cancer and cancer related disorders, comprise successive extracts of T. arjuna bark with ethyl acetate [AV016BaSu(65)09(100)], acetone [AV016BaSu(65)01(100)], ethanol [AV016BaSu(65)04(100)], methanol [AV016BaSu(65)06(100)] and water [AV016BaSu(105)08(100)], alone or in combination thereof. The pharmaceutical formulations may contain a pharmaceutically acceptable carrier, excipient, or diluent as outlined herein.

In a further aspect of the invention there is provided a comestible, that is to say, a foodstuff comprising at least an extract of the invention, typically in dried form, such as in a lyophilized form selected from those listed in Tables 1-24 herein, and in particular, from those extracts selected from those mentioned in Tables 25-30. The skilled addressee will appreciate that such comestibles may contain more than one extract of the invention and may be used. Such foodstuffs may be used in a prophylactic manner and may contain further extracts having a similar function to the first added extract or further added extracts may be added that have a different prophylactic function. Thus a foodstuff could either comprise extracts that provide for a comestible having a single functional aspect, for example that of having a prophylactic effect against the occurrence of diabetes, or a comestible may have a multi-functional prophylactic effect against two or more disease types. It is thought that a similar multi-functional role could also be assigned to pharmaceutical formulations comprising two or more extracts possessing dissimilar therapeutic or prophylactic properties designed either for prophylaxis or for the treatment of more than one disease(s) in a mammal, particularly in a human.

The type of foodstuff or comestible to which at least an extract of the invention may be added includes any processed food such as confectionaries, baked products including breads such as loafs, and flat breads such as pitta bread, naan bread and the like, cakes, snack foods such as muesli bars, compressed dried fruit bars, biscuits, dairy products such as yoghurts, milk and milk-based products such as custards, cream, cheese, butter and crème fraiche, simulated dairy food products such as margarine, olive oil-based spreads, and low fat cream substitutes such as Elmlea products, fruit and vegetable juices, aerated drinks, such as carbonated soft drinks and non-aerated drinks such as squashes, soya milk, rice milk and coconut milk and the like, pastas, noodles, vegetable, seed and nut oils, fruited oils such as sunflower oil, rapeseed oil, olive oil, walnut, hazelnut, and sesame seed oil and the like, and frozen confections such as ice creams, iced yoghurts and the like.

A suitable effective dose of an extract of the invention to be included in a comestible generally lies in the range of about 0.01 to about 120 mg/kg bodyweight, e.g. 0.1 to about 120 mg/kg body weight, preferably in the range of about 0.1 to 50 mg/kg, for example 0.5 to 50 mg/kg. The total daily dose may be given as a single dose, multiple doses, e.g. two to six times applications per day.

In a further aspect of the invention there is provided use of an extract selected from those of Tables 1-24, and in particular those of Tables 25-30 for the preparation of a medicament for the treatment of a disease selected from the group consisting of diabetes, cancer, cardiovascular disease and obesity.

Thus, there is provided use of an extract selected from the group consisting of AV016BaDi(65)04(100), AV016BaDi(28)04(20), AV016BaSu(65)09(100), AV016BaSu(65)01(100)_{g}, AV016BaSu(65)01(100)ng, AV016BaSu(65)04(100), AV016BaSu(65)06(100), AV016BaSu(105)08(100), AV016FrDi(65)04(100), and AV016FrSu(105)08(100) for the preparation of a medicament for the treatment or prophylaxis of diabetes and/or related diseases.

Additionally, there is provided use of an extract selected from the group consisting AV016BaDi(65)04(100), AV016BaDi(28)04(20), AV016BaSu(65)09(100), AV016BaSu(65)01(100)_{g}, AV016BaSu(65)01(100)ng, AV 016BaSu(65)04(100) and AV016BaSu(65)06(100), and AV016FrDi(65)04(100)) for the preparation of a medicament for the treatment or prophylaxis of obesity.

Additionally, there is provided use of an extract selected from the group consisting of AV016BaDi(65)04(100), AV016BaDi(28)04(20), AV016BaSu(65)09(100), AV016BaSu(65)01(100), either singly or in one or more combinations thereof for the preparation of a medicament for the treatment or prophylaxis of cardiovascular disease.

Additionally, there is provided use of an extract selected from the group consisting of AV016BaSu(65)09(100), AV016BaSu(65)01(100), AV016BaSu(65)04(100), AV016BaSu(65)06(100) and AV016BaSu(105)08(100), either singly or in one or more combinations thereof for the preparation of a medicament for the treatment or prophylaxis of cancer.

The invention will now be exemplified with reference to the following Examples section and accompanying tables and Figures. It is to be understood that the examples are not to be construed as limiting the scope of the invention in any way.

### EXAMPLES SECTION

### Example 1

### Extraction of Terminalia arjuna

Extraction of Terminalia arjuna plant parts was carried out by both direct extraction as well as successive extraction method, at room temperature as well as in soxhlet apparatus and related liquid-liquid techniques followed by lyophilization.

### I. Successive Extraction:

Successive extraction from bark of Terminalia arjuna was carried out using soxhlet extractor. The solvents used, were based on their sequential polarity starting from non-polar to polar, viz; Hexane, petroleum ether, chloroform, acetone, ethanol, methanol and water at 65° C.

The detailed process is given below:
1. Weigh 100 grams of powdered plant material into the extractor (Soxhlet extractor body) with the cotton on the bottom of the soxhlet apparatus. Cover with cotton on the top. Add 1000 ml of solvent (start with Hexane) in to the round-bottomed flask and place it on the mantle and add few ceramic chips in to it. Add 100 ml of solvent over the material just wetting it.
2. Place the extractor on the flask, which is in turn connected with the condenser.
3. Let the cold water circulate continuously in the condenser from the tap. The set up fits tightly as it is fabricated as one set.
4. Switch on the mantle and set it at 65° C. The vapors of the solvent from the flask would pass through the inlet of the extractor and will get condensed. The condensed (distilled) Solvent will get collected in the Extractor (body) thus extracting the compounds from it.
5. When the plant material is completely filled with solvents, it will get drained in the flask. This process is continuous as long as there is stable heat and water circulation.
6. Continue the extraction for 8 hours, 4-5 cycles per hour.
7. The extract collected in the flask is concentrated by vacuum lyophilization.
8. Follow the same procedure as above successively for the following solvents in the same order. Petroleum Ether, Ethyl acetate, Acetone, Ethanol, Methanol and Water.

### II. Direct Extraction

a. Soxhlet Based Extraction Procedure with 100% Ethanol Solvent:
1. Weigh 100 grams of powdered plant material in the cloth bag and transfer it into the extractor (Soxhlet extractor body). Cover with cotton on the top. (Make sure the level of material is below one inch of the vapour inlet tube.)
2. Add 1 liter of solvent (start with Pet, ether) in to the round-bottomed flask and place it on the mantle and add few ceramic chips in to it. Add 100 ml of solvent over the material just wetting it.
3. Place the extractor on the flask, which is in turn connected with the condenser.
4. Let the cold water circulate continuously in the condenser from the tap. The set up fits tightly as it is fabricated as one set.
5. Switch on the mantle and set it at 65° C. The vapours of the solvent from the flask would pass through the inlet of the extractor and will get condensed. The condensed (distilled) Solvent will get collected in the Extractor (body) thus extracting the compounds from it.
6. When the plant material is completely filled with solvents, it will get drained in the flask. This process is continuous as long as there is stable heat and water circulation.
7. Continue the extraction for 8 hours, 4-5 cycles per hour.
8. The extract collected in the flask is concentrated by vacuum lyophilization.
   b. Extraction of T. Arjuna Bark with 20% Ethanol Solvent at Room Temperature:
1. Weigh known quantity (100 grams) of powdered plant material into the conical flask and cover the mouth with aluminum foil to avoid solvent evaporation.
2. Add known volume (500 ml) of 20% ethanol (100 ml ethanol+400 ml water) solvent in to the flask and place it on to the orbital shaker and set the speed at 210 rpm and room 28° C. temperature for the extraction.
3. Extract the plant material for 4 hr and drain the solvent through
4. Centrifuge the filtrate at 1000 rpm for 10 mins. Collect the supernatant and subject it, to lyophilization.
5. Re-extract with 250 ml of solvent for (2×2 hrs).
6. Centrifuge the filtrate at 1000 rpm for 10 mins.
7. Concentrate extract using lyophilizer under vacuum.

### Example 2

### Metabolic Fingerprinting of the Terminalia arjuna Extracts

Metabolic fingerprinting of all the direct and successive extracts from Terminalia arjuna plant parts is done by HPLC and LC/MS technique.

### I. HPLC Fingerprinting:

The plant extracts obtained by direct/successive extraction are subjected to HPLC analysis. High Performance Liquid Chromatography (HPLC) is a technique wherein small quantity of the sample is injected into a C-18 column under high pressure and the constituents are allowed to separate based on their interaction with the column and their retention time within the column. The main purpose of HPLC analysis is to find out the total number of constituents in the plant extracts.

The samples are prepared for HPLC analysis by dissolving the appropriate weight of the extract in methanol. These samples are filtered and collected in the total recovery HPLC vials. These samples are subjected to separation by Waters 2695 HPLC instrument and then analyzed.

### Run Conditions:

1. The software used for HPLC analysis is Waters Millennium³².
2. The HPLC column used for separation is Waters μBondpack C-18, 5μ, 4.6×150 mm.
3. Column temperature is maintained at 25° C.
4. Solvent flow rate is set at 1.0 ml per min. HPLC conditions included Gradient chromatography—solvents used are acetonitrile (solvent A), methanol (solvent B) and water (Solvent C and D).

### Terminalia Arjuna Extracts and HPLC Run Conditions:

### 1. Terminalia Arjuna Extracts:

1. AV016BaDi(65)04(100)
2. AV016BaDi(28)04(20)
3. AV016BaSu(65)04(100)
4. AV016FrDi(65)04(100)
5. AV016BaSu(65)06(100)

I. Method Set: Ethanol_{—}11

Pressure Limits:
High Limits 4000 psi Low limits 0 psi

Programmed Flow:
Pump Mode: Gradient
Accelerated to 10 ml/min in: 2.0 min (5 ml/min/min)

| | Time | Flow | % A | % B | % C | % D | Curve |
|---|---|---|---|---|---|---|---|
| 1 | 0.01 | 1.00 | 10.0 | 0.0 | 0.0 | 90.0 | 6 |
| 2 | 1.00 | 1.00 | 10.0 | 0.0 | 0.0 | 90.0 | 6 |
| 3 | 15.00 | 1.00 | 30.0 | 0.0 | 0.0 | 70.0 | 6 |
| 4 | 30.00 | 1.00 | 40.0 | 0.0 | 0.0 | 60.0 | 6 |

A: Acetonitrile, B: Methanol, C: Water

### 2. Terminalia Arjuna Extracts

1. AV016BaSu(65)01(100)
2. AV016BaSu(65)01(100)g
3. AV016BaSu(65)01(100)g

II. Method Set: Ethyl Acetate_{—}10a

Pressure Limits:
High Limits 4000 psi Low limits 0 psi

Programmed Flow:
Pump Mode: Gradient
Accelerated to 10 ml/min in: 2.0 min (5 ml/min/min)

| | Time | Flow | % A | % B | % C | % D | Curve |
|---|---|---|---|---|---|---|---|
| 1 | 0.01 | 0.75 | 5.0 | 2.5 | 92.5 | 0.0 | 6 |
| 2 | 1.00 | 0.75 | 5.0 | 2.5 | 92.5 | 0.0 | 6 |
| 3 | 25.00 | 0.75 | 25.0 | 2.5 | 72.5 | 0.0 | 6 |
| 4 | 30.00 | 0.75 | 5.0 | 2.5 | 92.5 | 0.0 | 1 |

A: Acetonitrile, B: Methanol, C: Water

### 3. Terminalia Arjuna Extract:

1. AV016BaSu(65)09(100)

III. Method Set: Ethyl Acetate_{—}4a

Pressure Limits:
High Limits 4000 psi Low limits 0 psi

Programmed Flow:
Pump Mode: Gradient
Accelerated to 10 ml/min in: 2.0 min (5 ml/min/min)

| | Time | Flow | % A | % B | % C | % D | Curve |
|---|---|---|---|---|---|---|---|
| 1 | 0.01 | 0.75 | 0.0 | 5.0 | 95.0 | 0.0 | 6 |
| 2 | 1.00 | 0.75 | 0.0 | 5.0 | 95.0 | 0.0 | 6 |
| 3 | 15.00 | 0.75 | 0.0 | 20.0 | 80.0 | 0.0 | 6 |
| 4 | 25.00 | 0.75 | 0.0 | 50.0 | 50.0 | 0.0 | 6 |
| 5 | 30.00 | 0.75 | 0.0 | 5.0 | 95.0 | 0.0 | 1 |

A: Acetonitrile, B: Methanol, C: Water

### 4. Terminalia Arjuna Extract:

I. AV016BaSu(105)08(100)

2. AV016FrSu(105)08(100)

IV. Method Set: Gy_Water_{—}12

Pressure Limits:
High Limits 4000 psi Low limits 0 psi

Programmed Flow:
Pump Mode: Gradient
Accelerated to 10 ml/min in: 2.0 min (5 ml/min/min)

| | Time | Flow | % A | % B | % C | % D | Curve |
|---|---|---|---|---|---|---|---|
| 1 | 0.01 | 0.75 | 5.0 | 5.0 | 90.0 | 0.0 | 6 |
| 2 | 1.00 | 0.75 | 5.0 | 5.0 | 90.0 | 0.0 | 6 |
| 3 | 20.00 | 0.75 | 25.0 | 15.0 | 60.0 | 0.0 | 4 |
| 4 | 26.00 | 0.75 | 70.0 | 5.0 | 25.0 | 0.0 | 4 |
| 5 | 30.00 | 0.75 | 5.0 | 5.0 | 90.0 | 0.0 | 1 |

A: Acetonitrile, B: Methanol, C: Water

### II. Liquid Chromatography Mass Spectrometry (LC/MS) Fingerprinting:

Mass spectroscopy, is an instrumental approach that allows for the mass measurement of molecules. The five basic components of mass spectrometer are a vacuum system, a sample introduction device, an ionization source, a mass analyzer and an ion detector. Combining these parts a mass spectrometer determines the molecular weight of chemical compounds by ionizing, separating and measuring molecular ions according to their mass-to-charge ratio (m/z).

Run conditions used for LC/MS fingerprinting of Terminalia arjuna is shown down.
1. Q-Trap LC/MS instrument from Applied Biosystems was used. The software used for LC/MS analysis is Analyst.
2. The HPLC column used for separation is COSMOSIL® 5C₁₈-MS-II Packed Column C-18, 5 μm, 4.6 mmI.D.x 150 mm.
3. Column temperature is maintained at 25° C.
4. Solvent flow rate is set at 1.0 ml per min. HPLC conditions included Gradient chromatography—solvents used are acetonitrile (solvent C), methanol (solvent B) and water (Solvent D).

### 1. Terminalia Arjuna Extracts:

AV016BaSu(65)09(100), AV016BaSu(65)01(100), AV016BaSu(65)01(100)g, AV016BaSu(65)01(100)g, AV016BaSu(65)04(100), AV016BaSu(65)06(100), AV016BaSu(65)06(80), AV016BaDi(65)04(100), and AV016FrDi(65)04(100)

a. LC/MS Sample Run Conditions for all the Above-Mentioned Terminalia Arjuna Samples:

| | |
|---|---|
| Mass Spectrometer | QTrap 0 MASS SPEC |
| Config Table Ver | 01 |
| Firmware Ver | M401400 B4T0301 M3L1400 B3T0300 |
| Component Name | Linear Ion Trap Quadrupole |
| | LC/MS/MS Mass Spectrometer |
| Component ID | QTrap |
| Manufacturer | AB Sciex Instruments |
| Model | 027170 - C |
| S/N | M1100301 |
| Time from start = | 0.0500 min |
| Mass Spectrometer | Qtrap 0 MASS SPEC |
| Start of Run - Detailed Status | |
| Vacuum Status | At Pressure |
| Vacuum Gauge (10e−5 Torr) | 0.7 |
| Backing Pump | Ok |
| Dual Turbo Pump | Normal |
| Sample Introduction Status | Ready |
| Source/Ion Path Electronics | On |
| Source Type | Turbo Spray |
| Source Temperature (at setpoint) | 400.0 C. |
| Source Exhaust Pump | Ok |
| Interface Heater | Ready |
| Mass Spectrometer | Qtrap 0 MASS SPEC |
| End of Run - Detailed Status | |
| Vacuum Status | At Pressure |
| Vacuum Gauge (10e−5 Torr) | 0.7 |
| Backing Pump | Ok |
| Dual Turbo Pump | Normal |
| Sample Introduction Status | Ready |
| Source/Ion Path Electronics | On |
| Source Type | Turbo Spray |
| Source Temperature (at setpoint) | 400.0 C. |
| Source Exhaust Pump | Ok |
| Interface Heater | Ready |
| Time from start = | 61.4833 min |

### PE LC-200 Pump Method Properties

### PE LC-200 Quaternary Pump

Minimum Pressure (psi): 0.0
Maximum Pressure (psi): 6100.0
Shutdown Time (min): 999.9

### Step Table:

| | Total Time | Flow Rate | | | | | | | |
|---|---|---|---|---|---|---|---|---|---|
| Step | (min) | (μl/min) | GradientProfile | A (%) | B (%) | C (%) | D (%) | TE#1 | TE#2 |
| 0 | 0.5 | 1000.00 | 1.0 | 0.0 | 0.0 | 10.0 | 90.0 | open | open |
| 1 | 1.0 | 1000.00 | 1.0 | 0.0 | 0.0 | 10.0 | 90.0 | open | open |
| 2 | 15.0 | 1000.00 | 1.0 | 0.0 | 0.0 | 15.0 | 85.0 | open | open |
| 3 | 40.0 | 1000.00 | 1.0 | 0.0 | 0.0 | 25.0 | 75.0 | open | open |
| 4 | 50.0 | 1000.00 | 1.0 | 0.0 | 0.0 | 35.0 | 65.0 | open | open |
| 5 | 60.0 | 1000.00 | 1.0 | 0.0 | 0.0 | 50.0 | 50.0 | open | open |

### Quantitation Information:

### Sample Type Unknown

### Dilution Factor: 1.000000

### Custom Data:

### Quantitation Table:

### Period 1:

### Scans in Period: 2243

Relative Start Time: 0.00 msec

### Experiments in Period: 1

### Period 1 Experiment 1:

### Scan Type: Q1 MS (Q1)

### Polarity: Positive

### Scan Mode: Profile

### Resolution Q1: UNIT

### Intensity Thres.: 0.00 cps

### Settling Time: 0.0000 ms

### MR Pause: 5.0070 ms

### MCA: No

### Center/Width: No

### Step Size: 0.10 amu

| Start (amu) | Stop (amu) | Time (sec) | Param | Start | Stop |
|---|---|---|---|---|---|
| 50.00 | 1700.00 | 1.60 | CEP | 6.47 | 66.65 |

### Parameter Table (Period 1 Experiment 1)

### CUR: 20.00

### TEM: 400.00

### GS1: 20.00

### GS2: 50.00

### ihe: ON

### IS: 4500.00

### DP 90.00

### EP 10.00

### 2. Terminalia Arjuna Extracts:

AV016BaSu(105)08(100), AV016FrSu(105)08(100) and AV016BaDi(28)04(20).

a. LC/MS Sample Run Conditions for all the Above-Mentioned Terminalia Arjuna Samples:

| | |
|---|---|
| Mass Spectrometer | QTrap 0 MASS |
| SPEC | |
| Config Table Ver | 01 |
| Firmware Ver | M401400 B4T0301 M3L1400 B3T0300 |
| Component Name | Linear Ion Trap Quadrupole LC/MS/ |
| | MS Mass Spectrometer |
| Component ID | QTrap |
| Manufacturer | AB Sciex Instruments |
| Model | 027170-C |
| S/N | M1100301 |
| Time from start = 2.1000 min | |
| Mass Spectrometer | QTrap 0 MASS |
| SPEC | |
| Start of Run - Detailed Status | |
| Vacuum Status | At Pressure |
| Vacuum Gauge (10e−5 Torr) | 0.7 |
| Backing Pump | Ok |
| Dual Turbo Pump | Normal |
| Sample Introduction Status | Ready |
| Source/Ion Path Electronics | On |
| Source Type | Turbo Spray |
| Source Temperature (at setpoint) | 400.0 C. |
| Source Exhaust Pump | Ok |
| Interface Heater | Ready |
| Time from start = 2.1167 min | |
| Mass Spectrometer | QTrap 0 MASS |
| SPEC | |
| End of Run - Detailed Status | |
| Vacuum Status | At Pressure |
| Vacuum Gauge (10e−5 Torr) | 0.7 |
| Backing Pump | Ok |
| Dual Turbo Pump | Normal |
| Sample Introduction Status | Ready |
| Source/Ion Path Electronics | On |
| Source Type | Turbo Spray |
| Source Temperature (at setpoint) | 400.0 C. |
| Source Exhaust Pump | Ok |
| Interface Heater | Ready |
| Time from start = 42.9333 min | |

### PE LC-200 Pump Method Properties

### PE LC-200 Quaternary Pump

Minimum Pressure (psi): 0.0
Shutdown Time (min): 999.9

### Step Table:

| | Total Time | Flow Rate | | | | | | | |
|---|---|---|---|---|---|---|---|---|---|
| Step | (min) | (μl/min) | GradientProfile | A (%) | B (%) | C (%) | D (%) | TE#1 | TE#2 |
| 0 | 0.5 | 750.00 | 1.0 | 0.0 | 5.0 | 5.0 | 90.0 | open | open |
| 1 | 1.0 | 750.00 | 1.0 | 0.0 | 5.0 | 5.0 | 90.0 | open | open |
| 2 | 20.0 | 750.00 | 1.0 | 0.0 | 15.0 | 25.0 | 60.0 | open | open |
| 3 | 26.0 | 750.00 | 1.0 | 0.0 | 5.0 | 70.0 | 25.0 | open | open |
| 4 | 30.0 | 750.00 | 1.0 | 0.0 | 5.0 | 5.0 | 90.0 | open | open |
| 5 | 40.0 | 750.00 | 1.0 | 0.0 | 5.0 | 5.0 | 90.0 | open | open |

### Analog/Digital Converter Properties

Interval (sec): 0.200
Rate (pts/sec): 5

### Polarity: Bipolar

### Channel Summary

| | Name: | Interpreted Value Full Scale: | |
|---|---|---|---|
| No. | Voltage (volts): | Status: | Interpreted Unit: |
| 1 | 100.0 | % | 1.0 Used |

### Quantitation Information:

### Sample Type Unknown

### Dilution Factor: 1.000000

### Custom Data:

### Quantitation Table:

### Period 1:

### Scans in Period: 1495

Relative Start Time: 0.00 msec

### Experiments in Period: 1

### Period 1 Experiment 1:

### Scan Type: □1 MS (Q1)

### Polarity: Positive

### Scan Mode Profile

### Resolution Q1: UNIT

### Intensity Thres.: 0.00 cps

### Settling Time: 0.0000 ms

### MR Pause: 5.0070 ms

### MCA: No

### Center/Width: No

### Step Size: 0.10 amu

| Start (amu) | Stop (amu) | Time (sec) | Param | Start | Stop |
|---|---|---|---|---|---|
| 50.00 | 1700.00 | 1.60 | CEP | 6.47 | 66.65 |

### Parameter Table (Period 1 Experiment 1)

### CUR: 20.00

### TEM: 400.00

### GS1: 20.00

### GS2: 50.00

### ihe: ON

### IS: 4500.00

### DP 90.00

### EP 10.00

### Example 3

### Determination of the Bio-Therapeutic Potential of Terminalia Arjuna Extracts

### I. Anti-Diabetic Potential:

Acute glucose elevations after meal ingestion are associated with a variety of glucose-mediated tissue defects oxidative stress, glycation, and advanced glycation end product formation which have far reaching structural and functional consequences for virtually every human organ system. Lowering glycosylated hemoglobin to levels that prevent or delay these complications can be achieved only by reducing both postprandial and fasting plasma glucose levels.

The α-Glucosidase complex in the small intestine is involved in sugar absorption. Thus, inhibitors of α-Glucosidase would limit the absorption of dietary carbohydrates and in turn suppress postprandial hyperglycemia. Therefore, α-Glucosidase inhibitors are promising drug candidates in the treatment and prevention of diabetes. The α-Glucosidase inhibitors (acarbose, voglibose, miglitol) have been effective in delaying the digestion and absorption of carbohydrates, thus diminishing the postprandial surge in blood glucose levels without loss of calories.

### 1. Alpha-Glucosidase Assay

Alpha-Glucosidase hydrolyzes the terminal, non-reducing 1,4-linked alpha-D-glucose residues with release of alpha-D-glucose. In the assay the enzymatic activity is determined by measuring the increase in absorbance at 400 nm caused by the hydrolysis of p-nitrophenyl-α-D-glucopyranoside with the release of p-nitrophenol.

a. Reagents:
Potassium phosphate buffer 0.1M, pH 6.8
PNPG 4 mM
Sodium carbonate 0.2 M
BSA 0.2% in Buffer
Enzyme 1.2 U/ml in 0.2% BSA
   b. Procedure:

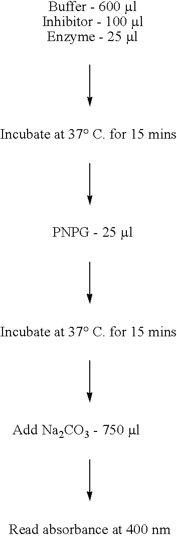

c. Results and Discussion:

Successive and direct extract from Terminalia arjuna bark and fruit extracts assayed for their alpha-Glucosidase inhibition potential showed that T. arjuna extract showed dose dependent decrease in alpha-Glucosidase inhibition. IC₅₀ is defined as the amount of extract required for 50% inhibition in the enzyme activity.

It was found that all the extracts of T. arjuna showed higher alpha-Glucosidase inhibition potential than commercially available acarbose, as indicated by IC₅₀ values. (Table 25).

### 2 Glucose Uptake Assay:

The deoxy-glucose uptake assay by fat cells or adipoctes (3T3-L1) help to identify the extracts/bioactives that helps to increase the glucose uptake by the fat cells. Differentiating 3T3-L1 cells exhibit a dramatic increase in the rate of insulin-stimulated glucose transport during their conversion from proliferating fibroblasts to nonproliferating adipocytes. Green and Kehinde (1975) first demonstrated that confluent 3T3-L1 cells could be hormonally induced to differentiate into adipocytes over the course of 6-9 d.

The assay will thereby help in screening of phytoextracts that will help the diabetic individuals by quick removal of glucose from blood stream and simultaneously will help the fat cells to build a better storage of energy.

Tritiated deoxyglucose uptake by 3T3-L1 is one of the most accepted assay for the in-vitro screening of active principles with anti-diabetic activity

### a. 3T3-L1 Cell Culture:

3T3-L1 fibroblasts were cultured in DME-F12 media containing 10% FCS, NEAA, glutamine, antibiotics, anti-mycotics, etc., in an atmosphere of 5% CO2 at 37° C. Fibroblasts were cultured up to confluency. Subcultures were done at three-day intervals with trypsin-EDTA solution treatment.

b. Maturation of the Fibroblasts to Adipocytes

Differentiation was induced by treating the cells with DMEM hi glucose (4.5 gm/litre) containing 0.5 mmol/l 3-isobutyl-1-methylxanthine, 4 mg/ml dexamethasone, 1 mg/ml insulin and 10% FCS for 48 h. The cells were cultured in DMEM media with standard pre-described supplements for the next 6-10 days. Cells were used only when at least 95% of the cells showed an adipocyte phenotype by accumulation of lipid droplets. These were taken up for glucose uptake studies with tritiated glucose.

c. Assay Procedure

Distribute 50,000 of differentiated 3T3-L1 adipocytes in each well of a new 96 well plate. Incubate with KRH buffer at 37° C. for 10 min. Add insulin 10 nM, 25 nM and 50 nM to the respective. To the experimental sets treatment was done with or without insulin 10 nM in presence of test compound (333.0 μg). This was followed by incubation further at 37° C. for 15 min. Wash the cells once with KRH buffer. Initiate glucose uptake reaction by adding 0.1 mM 2-deoxy glucose containing 2-deoxy[3H] glucose (final concentration 12.2 kBq/ml). Incubate for 1 hr at 37° C. Terminate assay by adding 40 μM Cytochalasin B. Wash the cells trice with ice-cold KRH buffer Solubilize cells in 20 μl 1% Triton X. Incubate for 10 min at 37° C. Count radioactivity on a micro-titre plate radioactive counter

d. Results and Discussion:

Glucose uptake activity was analyzed by measuring the uptake of 0.1 mM deoxy-glucose containing deoxy-tritiated-glucose containing 2.2 kBq/mL.

As seen from Table 26, direct extract of T. arjuna bark with 100% ethanol solvent [AV016BaDi(65)04(100)] showed stimulation index of 3.0 in absence of insulin and 2.0 in presence of 10 nM Insulin. The data thus suggest that AV016BaDi(65)04(100) has potent insulin mimetic potential.

### 2. Anti-Obesity Potential:

Studies have demonstrated that central obesity is more strongly related to lipid/lipoprotein abnormalities than is general obesity. Obesity may confer cardiac dysfunction due to lipid accumulation in cardiomyocytes and also induces type II diabetes. These adverse lipid/lipoprotein profiles in obese individuals are important, because they may be responsible for their increased risk for cardiovascular disease (CVD).

The assay is a semi quantitative method used for quantification of Adipose differentiation and hence the lipid and triglycerides contents in the cell after the stimulation of the cells for differentiation in presence and absence of the plant extracts.

a. Assay Principle

Oil Red staining is a semi quantitative method used for quantification of Adipose differentiation. Staining with oil-soluble dyes is based on the greater solubility of the dye in the lipoid substances than in the usual hydroalcoholic dye solvents. Oil Red 0 is a lipophilic red dye which stains intracellular lipid drops. The quantitation of lipid can be done following the extraction of intracellular lipid with organic solvent. Hence it is used to estimate lipid and triglycerides contents in the cell after plant extract stimulation of cells

b. Assay Protocol

Seed 3T3-L1 cells at a density of 10⁵ cells/well with the differentiating medium and extracts (0.33 mg) and incubated for 24 h at 37° C. and 5% CO₂. The medium was then removed after 24 hr and fresh differentiating medium was added and the cells were kept in the medium for another 24 hrs. Post 48 hrs from day one the medium was removed and Insulin only medium (DMEM F12+FBS+Insulin) was added and the cells were kept for 24 h. Post 24-hr incubation medium was aspirated and Oil O red staining was performed.

c. Oil Red O Staining:

Total volume in each well for reagents is 500 μl/well. Media was removed and cells were washed with PBS thrice. For fixing the cells they were incubated for 1 h with 4% Formalin. Remove the Formalin and again wash with PBS twice. For staining with Oil Red O, incubate it with the stain for 1 h. After 1 h remove the stain and wash it with PBS and dry it for 10 minutes. Add 4% NP40. Read absorbance at 520 nm in Spectrophotometer.

d. Results and Discussion:

Anti-obesity assay with 3T3-L1 cells showed that Terminalia arjuna extracts showed inhibition in the lipid accumulation. As seen from Table 27 Terminalia arjuna bark direct 100% and 20% ethanol extracts, AV016BaDi(65)04(100) and AV016BaDi(28)04(20) extracts showed 76.3 and 64.3% inhibition as compared to control.

In case of Terminalia arjuna successive bark ethyl acetate, gluey acetone, non-gluey acetone, ethanol and methanol extracts AV016BaSu(65)09(100), AV016BaSu(65)01(100)_{g}, AV016BaSu(65)01(100)ng, AV016BaSu(65)04(100) and AV016BaSu(65)06(100) gave inhibition of 61.9, 58.5, 86.8, 49.4 and 57.9% inhibition as compared to control. Terminalia arjuna fruit direct 100% ethanol extract AV016FrDi(65)04(100) gave inhibition of 41.9% inhibition as compared to control.

Assay+ve control showed 104% inhibition as compared to control.

### 3. Cardiovascular Disease:

Elevated cholesterol is associated with a greater-than-normal risk of atherosclerosis and cardiovascular disease. High blood triglyceride levels alone do not cause atherosclerosis. But lipoproteins that are rich in triglycerides also contain cholesterol, which causes atherosclerosis in many people with high triglycerides. So high triglycerides may be a sign of a lipoprotein problem that contributes to CHD.

### 1. Cell Based Assay for Checking Levels of Lipid Accumulation:

a. Assay Principle:

Growing the cells in presence of lipoprotein deficient serum induces the lipid biosynthesis in the cells. Assay is performed on the cells induced by growing in lipoprotein deficient serum followed by total lipid estimation using oil red staining. Oil Red O is a lipophilic red dye used to stain neutral lipids in cells. The amount of staining is directly proportional to the amount of lipid in the cell. The content of lipid was checked across the extract treated and control sets and the amount of dye and hence lipid content was measured spectrophotometrically.

b. Assay Procedure

CHO-K1 cells were seeded at density of 10 cells/well in 12 well-plate format. The cells were grown to 80% confluence in DMEM-F12 medium with 10% fetal calf serum, 2 mM glutamine, 1.5 gm/L sodium bicarbonate and anti-mycotics and penicillin and streptomycin at 37 C in 5% CO2 environment. After 2 days of incubation the medium was replaced with DMEM-F 12 medium with 10% lipoprotein deficient serum, 2 mM glutamine, 1.5 gm/L sodium bicarbonate and anti-mycotics and penicillin and streptomycin and grown for additional 24 hrs at 37 C in 5% CO2 environment. At the end of 24 hrs to the extract treated sets Terminalia arjuna at concentration of 0.5 mg/ml were added whereas the control sets were treated with vehicle (DMSO). The incubation was continued for additional 4 hrs after which the cells were washed with sterile PBS buffer, fixed and stained with Oil Red O.

c. Oil Red O Staining:

Total volume in each well for reagents is 500 μl/well. Media was removed and cells were washed with PBS thrice. For fixing the cells they were incubated for 1 h with 4% Formalin. Remove the Formalin and again wash with PBS twice. For staining with Oil Red O, incubate it with the stain for 1 h. After 1 h remove the stain and wash it with PBS and dry it for 10 minutes. Add 4% NP40. Read absorbance at 520 nm in Spectrophotometer.

d. Results and Discussion:

As seen from Table 28, Terminalia arjuna extracts showed marked decrease in the amount of total lipids in the extract treated cells. Terminalia arjuna bark successive extracts AV016BaSu(65)09(100) and AV016BaSu(65)01(100) showed 22% and 21% inhibition as compared to control set. Terminalia arjuna direct ethanol extracts AV016BaDi(65)04(100) and AV016BaDi(28)04(20) showed 31% and 28% inhibition in the levels of total lipids as compared to the control set.

### 2. HMG-CoA Reductase Assay:

3-Hydroxy-3-methylglutaryl-coenzyme A (HMG-CoA) reductase is the first committed step in cholesterol biosynthetic pathway. HMG-CoA reductase the rate-limiting enzyme step in cholesterol biosynthesis has become the most successful drug treatment for lowering total plasma cholesterol, in particular LDL-cholesterol.

a. Assay Principle:

HMG-CoA reductase assay is based on the oxidation of NADPH to NADP catalyzed by a fixed concentration of HMG-CoA reductase from liver microsomal fraction.

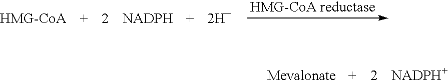

The oxidation of NADPH to NADP⁺ is accompanied by a decrease in absorbance at 340 nm and is directly proportional to the HMG-CoA reductase activity in the sample.

b. Results and Discussion:

The potential of Terminalia arjuna bark direct 100% ethanol and 20% ethanol extracts AV016BaDi(28)04(20) and AV016BaDi(65)04(100) were checked for their HMG-CoA reductase inhibition potential. It was found that both AV016BaDi(28)04(20) and AV016BaDi(65)04(100) showed 93% inhibition (Table 29) and 63.1% (Table 30) in HMG-CoA reductase activity as compared to control set.

### 4. Anti-Cancer Assay:

The Anti-cancer potential of the Terminalia arjuna plant extracts was determined by screening the plant extracts in vitro for cytotoxicity on the human hepatic cell line HepG2.

a. Experimental Details:

The cells were seeded at a density of 2.5×10⁵ cell per ml in a 24 well plate. The cells were allowed to attach for 2 hour before adding the extracts at a concentration of 10 μg/ml. 48 hour after incubation with vehicle i.e DMSO, ascorbic acid and extracts AV016BaSu(65)09(100), AV016BaSu(65)01(100), AV016BaSu(65)04(100). The cell viability was determined after 24 hours.

b. Assay Protocol

Seed HepG2 at a cell density of 2.5×10⁵ cells per ml. Add of the Ascorbic acid and Terminalia arjuna extracts to final concentration of 10 μg/ml. Incubate in 5% CO₂ incubator at 37° C. for 48 hour. Trypsinize cell. Mix with 1 part trypan blue and 4 parts PBS and count the viable cells and the non-viable cells by counting the cells that has taken typan blue (non-viable cells) and the ones that has excluded it (viable cells).

c. Results and Discussions:

It was found that treatment of HepG2 cells with T. arjuna successive ethyl acetate AV016BaSu(65)09(100), acetone AV016BaSu(65)01(100), and ethanol AV016BaSu(65)04(100), extracts there was decrease in the total cell count and in the number of viable cells in case of drug treated set as compared to the untreated set or the cells with DMSO set (Table 31).

TABLE 1
HPLC fingerprint of extract AV016BaDi(65)04(100)

| | Retention | | | |
|---|---|---|---|---|
| | Time | Area | % Area | Height |
| 1 | 1.746 | 83501 | 0.03 | 18746 |
| 2 | 1.887 | 21939706 | 9.17 | 3508953 |
| 3 | 2.482 | 424664 | 0.18 | 27549 |
| 4 | 3.165 | 694426 | 0.29 | 38196 |
| 5 | 4.499 | 535430 | 0.22 | 20883 |
| 6 | 5.860 | 1140766 | 0.48 | 31447 |
| 7 | 6.488 | 2287279 | 0.96 | 105829 |
| 8 | 7.680 | 2247351 | 0.94 | 73739 |
| 9 | 8.259 | 1172094 | 0.49 | 53148 |
| 10 | 8.702 | 2209397 | 0.92 | 93831 |
| 11 | 9.425 | 2269048 | 0.95 | 89553 |
| 12 | 10.177 | 3848206 | 1.61 | 111334 |
| 13 | 10.485 | 1536103 | 0.64 | 99241 |
| 14 | 10.730 | 1306318 | 0.55 | 102448 |
| 15 | 11.155 | 3705535 | 1.55 | 129030 |
| 16 | 12.263 | 10146875 | 4.24 | 464227 |
| 17 | 12.335 | 6747979 | 2.82 | 548102 |
| 18 | 13.000 | 30474340 | 12.74 | 1778085 |
| 19 | 13.697 | 3437393 | 1.44 | 233361 |
| 20 | 14.232 | 8223596 | 3.44 | 277375 |
| 21 | 14.532 | 14638851 | 6.12 | 819721 |
| 22 | 15.023 | 12657916 | 5.29 | 590365 |
| 23 | 15.452 | 5471266 | 2.29 | 306520 |
| 24 | 15.998 | 16599596 | 6.94 | 1078704 |
| 25 | 16.299 | 4397452 | 1.84 | 318952 |
| 26 | 16.463 | 2424066 | 1.01 | 310668 |
| 27 | 16.863 | 6414264 | 2.68 | 310396 |
| 28 | 17.166 | 12652492 | 5.29 | 355032 |
| 29 | 17.683 | 4940680 | 2.07 | 294832 |
| 30 | 18.118 | 11457746 | 4.79 | 318670 |
| 31 | 18.673 | 2951869 | 1.23 | 232444 |
| 32 | 18.872 | 6643592 | 2.78 | 219376 |
| 33 | 19.475 | 1933618 | 0.81 | 176183 |
| 34 | 19.668 | 2454443 | 1.03 | 182659 |
| 35 | 19.884 | 4858876 | 2.03 | 167366 |
| 36 | 20.329 | 950214 | 0.40 | 139162 |
| 37 | 20.543 | 6254360 | 2.61 | 134329 |
| 38 | 21.427 | 1804592 | 0.75 | 107259 |
| 39 | 21.716 | 7198552 | 3.01 | 109172 |
| 40 | 23.185 | 2885830 | 1.21 | 72715 |
| 41 | 23.905 | 3042057 | 1.27 | 54167 |
| 42 | 24.977 | 665797 | 0.28 | 38055 |
| 43 | 25.258 | 1486354 | 0.62 | 35170 |

TABLE 2
HPLC fingerprint of extract AV016BaDi(28)04(20).

| | Retention | | | |
|---|---|---|---|---|
| | Time | Area | % Area | Height |
| 1 | 1.724 | 259750 | 0.10 | 58489 |
| 2 | 1.897 | 27826788 | 10.53 | 3799266 |
| 3 | 2.486 | 571316 | 0.22 | 50306 |
| 4 | 3.045 | 1106233 | 0.42 | 51910 |
| 5 | 3.732 | 674291 | 0.26 | 29021 |
| 6 | 4.182 | 1056425 | 0.40 | 32454 |
| 7 | 5.572 | 2403677 | 0.91 | 47788 |
| 8 | 6.082 | 3557804 | 1.35 | 161895 |
| 9 | 6.484 | 757991 | 0.29 | 55308 |
| 10 | 7.264 | 3549212 | 1.34 | 97606 |
| 11 | 7.883 | 2512425 | 0.95 | 92337 |
| 12 | 8.299 | 3676625 | 1.39 | 140954 |
| 13 | 9.060 | 4300244 | 1.63 | 140449 |
| 14 | 9.699 | 6186918 | 2.34 | 155916 |
| 15 | 10.124 | 2085208 | 0.79 | 151077 |
| 16 | 10.796 | 7036369 | 2.66 | 179944 |
| 17 | 11.555 | 17109248 | 6.48 | 350960 |
| 18 | 12.574 | 31568344 | 11.95 | 1286226 |
| 19 | 13.038 | 2562494 | 0.97 | 285400 |
| 20 | 13.307 | 4871494 | 1.84 | 288682 |
| 21 | 14.002 | 26201944 | 9.92 | 838854 |
| 22 | 14.597 | 14156853 | 5.36 | 465017 |
| 23 | 15.150 | 4497591 | 1.70 | 322199 |
| 24 | 15.663 | 16422598 | 6.22 | 962872 |
| 25 | 15.912 | 3863537 | 1.46 | 326789 |
| 26 | 16.103 | 6416104 | 2.43 | 315587 |
| 27 | 16.564 | 4817209 | 1.82 | 305649 |
| 28 | 16.767 | 8560395 | 3.24 | 305526 |
| 29 | 17.221 | 2824887 | 1.07 | 283965 |
| 30 | 17.400 | 2813123 | 1.06 | 282612 |
| 31 | 17.661 | 16467191 | 6.23 | 382391 |
| 32 | 18.625 | 6447644 | 2.44 | 200677 |
| 33 | 19.302 | 3348258 | 1.27 | 168434 |
| 34 | 19.568 | 11717199 | 4.44 | 149595 |
| 35 | 21.405 | 4945619 | 1.87 | 87932 |
| 36 | 22.629 | 4393424 | 1.66 | 69440 |
| 37 | 24.162 | 2305522 | 0.87 | 34152 |
| 38 | 25.963 | 255400 | 0.10 | 9559 |

TABLE 3
HPLC fingerprint of extract AV016BaSu(65)09(100).

| | Retention | | | |
|---|---|---|---|---|
| | Time | Area | % Area | Height |
| 1 | 2.475 | 25395907 | 5.13 | 3334075 |
| 2 | 3.244 | 1688929 | 0.34 | 136464 |
| 3 | 4.656 | 9262251 | 1.87 | 280029 |
| 4 | 5.254 | 1188708 | 0.24 | 48754 |
| 5 | 6.603 | 12296585 | 2.48 | 364675 |
| 6 | 6.899 | 5848912 | 1.18 | 389909 |
| 7 | 7.233 | 1367772 | 0.28 | 130776 |
| 8 | 7.428 | 1332175 | 0.27 | 138611 |
| 9 | 7.896 | 7084034 | 1.43 | 280115 |
| 10 | 8.260 | 6705156 | 1.35 | 380995 |
| 11 | 8.576 | 2924953 | 0.59 | 246136 |
| 12 | 8.782 | 2453519 | 0.50 | 228061 |
| 13 | 9.184 | 8669542 | 1.75 | 320819 |
| 14 | 9.521 | 4295969 | 0.87 | 304404 |
| 15 | 9.816 | 4279516 | 0.86 | 324956 |
| 16 | 10.243 | 30792046 | 6.22 | 3248747 |
| 17 | 10.516 | 84937402 | 17.16 | 4333685 |
| 18 | 11.442 | 12774541 | 2.58 | 1056539 |
| 19 | 11.603 | 33723533 | 6.81 | 4613519 |
| 20 | 11.705 | 63394649 | 12.81 | 4142892 |
| 21 | 12.316 | 44870739 | 9.06 | 3991785 |
| 22 | 12.575 | 5106719 | 1.03 | 582499 |
| 23 | 12.754 | 2812210 | 0.57 | 307827 |
| 24 | 12.983 | 7556632 | 1.53 | 836964 |
| 25 | 13.270 | 4609110 | 0.93 | 360754 |
| 26 | 13.418 | 4222758 | 0.85 | 346063 |
| 27 | 13.925 | 16253486 | 3.28 | 1431902 |
| 28 | 14.456 | 7163578 | 1.45 | 605726 |
| 29 | 14.797 | 1631474 | 0.33 | 141283 |
| 30 | 15.240 | 3874910 | 0.78 | 285386 |
| 31 | 15.388 | 3029615 | 0.61 | 225745 |
| 32 | 15.588 | 2719921 | 0.55 | 159504 |
| 33 | 16.139 | 1333368 | 0.27 | 123152 |
| 34 | 16.346 | 3614771 | 0.73 | 345601 |
| 35 | 16.570 | 1244982 | 0.25 | 111424 |
| 36 | 16.813 | 1256674 | 0.25 | 123386 |
| 37 | 16.995 | 1494705 | 0.30 | 110575 |
| 38 | 17.367 | 1278577 | 0.26 | 73925 |
| 39 | 17.823 | 2517030 | 0.51 | 117093 |
| 40 | 18.123 | 2322326 | 0.47 | 179787 |
| 41 | 18.419 | 841112 | 0.17 | 86329 |
| 42 | 18.673 | 2150256 | 0.43 | 93219 |
| 43 | 18.954 | 429553 | 0.09 | 73333 |
| 44 | 19.255 | 1852803 | 0.37 | 78786 |
| 45 | 19.627 | 1385755 | 0.28 | 84299 |
| 46 | 19.912 | 1175927 | 0.24 | 106435 |
| 47 | 20.169 | 2238619 | 0.45 | 198261 |
| 48 | 20.362 | 1903752 | 0.38 | 136402 |
| 49 | 20.725 | 1388092 | 0.28 | 86158 |
| 50 | 21.002 | 2038182 | 0.41 | 112055 |
| 51 | 21.221 | 957990 | 0.19 | 84615 |
| 52 | 21.507 | 1049496 | 0.21 | 79603 |
| 53 | 21.799 | 1772753 | 0.36 | 102185 |
| 54 | 22.203 | 1643957 | 0.33 | 76435 |
| 55 | 22.543 | 831012 | 0.17 | 71872 |
| 56 | 22.690 | 572833 | 0.12 | 72239 |
| 57 | 22.941 | 3535908 | 0.71 | 220985 |
| 58 | 23.299 | 2133293 | 0.43 | 142496 |
| 59 | 23.648 | 2144927 | 0.43 | 151460 |
| 60 | 23.960 | 1493722 | 0.30 | 100416 |
| 61 | 24.429 | 2297448 | 0.46 | 123572 |
| 62 | 24.639 | 908084 | 0.18 | 85150 |
| 63 | 24.933 | 2402551 | 0.49 | 104466 |
| 64 | 25.280 | 583388 | 0.12 | 83878 |
| 65 | 25.564 | 1925541 | 0.39 | 92355 |
| 66 | 25.800 | 1153719 | 0.23 | 90624 |
| 67 | 25.974 | 1727846 | 0.35 | 88759 |
| 68 | 26.404 | 1397501 | 0.28 | 91036 |
| 69 | 26.591 | 795071 | 0.16 | 89416 |
| 70 | 26.751 | 1482526 | 0.30 | 89528 |
| 71 | 27.018 | 763878 | 0.15 | 86118 |
| 72 | 27.157 | 1700407 | 0.34 | 86915 |
| 73 | 27.647 | 2637337 | 0.53 | 78919 |
| 74 | 28.193 | 4360949 | 0.88 | 662358 |

TABLE 4
HPLC fingerprint of extract AV016BaSu(65)01(100).

| | Retention | | | |
|---|---|---|---|---|
| | Time | Area | % Area | Height |
| 1 | 2.391 | 6928 | 0.00 | 852 |
| 2 | 2.556 | 1739539 | 0.82 | 304280 |
| 3 | 3.313 | 84646 | 0.04 | 9680 |
| 4 | 3.686 | 223139 | 0.10 | 9176 |
| 5 | 4.269 | 63325 | 0.03 | 3419 |
| 6 | 5.024 | 13893 | 0.01 | 769 |
| 7 | 6.157 | 4281 | 0.00 | 572 |
| 8 | 6.736 | 197041 | 0.09 | 6916 |
| 9 | 7.391 | 117641 | 0.06 | 5898 |
| 10 | 7.878 | 616604 | 0.29 | 17813 |
| 11 | 8.308 | 278599 | 0.13 | 15829 |
| 12 | 8.860 | 611104 | 0.29 | 22047 |
| 13 | 9.391 | 187896 | 0.09 | 12237 |
| 14 | 9.564 | 276548 | 0.13 | 14167 |
| 15 | 12.058 | 8846367 | 4.16 | 183309 |
| 16 | 12.644 | 751247 | 0.35 | 38688 |
| 17 | 13.199 | 1719360 | 0.81 | 43466 |
| 18 | 13.988 | 1735451 | 0.82 | 56406 |
| 19 | 14.555 | 2169673 | 1.02 | 76948 |
| 20 | 14.875 | 2254289 | 1.06 | 115963 |
| 21 | 15.626 | 4155110 | 1.95 | 123796 |
| 22 | 15.956 | 1080279 | 0.51 | 86186 |
| 23 | 16.580 | 5912054 | 2.78 | 175797 |
| 24 | 17.766 | 7200465 | 3.38 | 141141 |
| 25 | 18.423 | 2986827 | 1.40 | 111890 |
| 26 | 19.067 | 6971841 | 3.28 | 151382 |
| 27 | 19.737 | 4017460 | 1.89 | 145309 |
| 28 | 20.104 | 1488984 | 0.70 | 136621 |
| 29 | 20.459 | 4173594 | 1.96 | 175505 |
| 30 | 20.795 | 3235535 | 1.52 | 167773 |
| 31 | 21.079 | 1788929 | 0.84 | 164753 |
| 32 | 21.272 | 4796914 | 2.25 | 175363 |
| 33 | 21.910 | 4124406 | 1.94 | 168234 |
| 34 | 22.267 | 6071222 | 2.85 | 185861 |
| 35 | 22.997 | 5930428 | 2.79 | 190259 |
| 36 | 23.451 | 5868309 | 2.76 | 231002 |
| 37 | 23.866 | 16287689 | 7.65 | 1076501 |
| 38 | 24.287 | 4035419 | 1.90 | 266032 |
| 39 | 24.627 | 3569391 | 1.68 | 266672 |
| 40 | 24.844 | 42015183 | 19.75 | 2046204 |
| 41 | 26.107 | 8757964 | 4.12 | 239370 |
| 42 | 26.900 | 25586681 | 12.02 | 821474 |
| 43 | 27.926 | 7429374 | 3.49 | 323025 |
| 44 | 28.156 | 13400123 | 6.30 | 654328 |

TABLE 5
HPLC fingerprint of extract AV016BaSu(65)01(100)ng.

| | Retention | | | |
|---|---|---|---|---|
| | Time | Area | % Area | Height |
| 1 | 2.579 | 1411822 | 0.59 | 207187 |
| 2 | 3.336 | 80911 | 0.03 | 9988 |
| 3 | 3.518 | 367100 | 0.15 | 14428 |
| 4 | 4.289 | 54629 | 0.02 | 3130 |
| 5 | 4.766 | 46285 | 0.02 | 1682 |
| 6 | 6.659 | 58963 | 0.02 | 2489 |
| 7 | 7.316 | 3635 | 0.00 | 454 |
| 8 | 7.852 | 277194 | 0.12 | 10198 |
| 9 | 8.240 | 150308 | 0.06 | 8971 |
| 10 | 8.774 | 321493 | 0.14 | 13773 |
| 11 | 9.207 | 113505 | 0.05 | 7075 |
| 12 | 9.458 | 151700 | 0.06 | 8682 |
| 13 | 10.403 | 841535 | 0.35 | 23389 |
| 14 | 11.877 | 5609438 | 2.36 | 151067 |
| 15 | 12.469 | 548895 | 0.23 | 28503 |
| 16 | 12.997 | 1315579 | 0.55 | 34524 |
| 17 | 13.798 | 1396765 | 0.59 | 46557 |
| 18 | 14.301 | 1552115 | 0.65 | 56533 |
| 19 | 14.658 | 1693118 | 0.71 | 88234 |
| 20 | 15.378 | 2309177 | 0.97 | 76351 |
| 21 | 15.719 | 1849902 | 0.78 | 89567 |
| 22 | 16.341 | 3751422 | 1.58 | 131368 |
| 23 | 16.651 | 705622 | 0.30 | 59259 |
| 24 | 17.537 | 5824011 | 2.45 | 121156 |
| 25 | 18.170 | 2202916 | 0.93 | 92276 |
| 26 | 19.153 | 16377423 | 6.89 | 384476 |
| 27 | 20.514 | 11154160 | 4.69 | 206032 |
| 28 | 21.248 | 27468304 | 11.56 | 1141614 |
| 29 | 22.005 | 3503203 | 1.47 | 178231 |
| 30 | 22.665 | 5722669 | 2.41 | 184191 |
| 31 | 23.185 | 15337448 | 6.45 | 535651 |
| 32 | 23.666 | 13965998 | 5.88 | 926816 |
| 33 | 24.155 | 2815614 | 1.18 | 263179 |
| 34 | 24.360 | 4975496 | 2.09 | 287271 |
| 35 | 24.677 | 30074237 | 12.65 | 1853497 |
| 36 | 25.316 | 9050757 | 3.81 | 411682 |
| 37 | 25.712 | 6189308 | 2.60 | 264816 |
| 38 | 26.607 | 20990123 | 8.83 | 826640 |
| 39 | 26.996 | 4842260 | 2.04 | 310728 |
| 40 | 27.520 | 16829287 | 7.08 | 814850 |
| 41 | 27.942 | 15732031 | 6.62 | 542909 |

TABLE 6
HPLC fingerprint of extract AV016BaSu(65)01(100)g.

| | Retention | | | |
|---|---|---|---|---|
| | Time | Area | % Area | Height |
| 1 | 2.384 | 12028 | 0.01 | 947 |
| 2 | 2.570 | 2633549 | 1.23 | 435477 |
| 3 | 3.507 | 735108 | 0.34 | 33546 |
| 4 | 5.043 | 9805 | 0.00 | 651 |
| 5 | 6.822 | 78175 | 0.04 | 3779 |
| 6 | 7.045 | 83478 | 0.04 | 5089 |
| 7 | 7.203 | 66115 | 0.03 | 5355 |
| 8 | 7.820 | 581086 | 0.27 | 16595 |
| 9 | 8.749 | 775713 | 0.36 | 25475 |
| 10 | 9.447 | 325440 | 0.15 | 11555 |
| 11 | 10.387 | 882547 | 0.41 | 20400 |
| 12 | 11.017 | 953840 | 0.45 | 28086 |
| 13 | 11.853 | 2915239 | 1.36 | 86837 |
| 14 | 12.467 | 565867 | 0.26 | 27905 |
| 15 | 13.088 | 924183 | 0.43 | 31678 |
| 16 | 13.160 | 468207 | 0.22 | 31979 |
| 17 | 13.766 | 1224906 | 0.57 | 43299 |
| 18 | 14.633 | 3503010 | 1.64 | 84712 |
| 19 | 15.471 | 1966123 | 0.92 | 60085 |
| 20 | 15.728 | 1016548 | 0.48 | 66817 |
| 21 | 16.317 | 3374916 | 1.58 | 110591 |
| 22 | 16.627 | 663866 | 0.31 | 60558 |
| 23 | 17.526 | 4303710 | 2.01 | 88642 |
| 24 | 17.688 | 741644 | 0.35 | 86630 |
| 25 | 18.026 | 1554964 | 0.73 | 90925 |
| 26 | 18.356 | 1800352 | 0.84 | 101584 |
| 27 | 18.852 | 3360093 | 1.57 | 117653 |
| 28 | 19.607 | 6271811 | 2.93 | 186866 |
| 29 | 19.840 | 4324472 | 2.02 | 201700 |
| 30 | 20.208 | 6132533 | 2.87 | 247918 |
| 31 | 20.820 | 2351992 | 1.10 | 151443 |
| 32 | 21.075 | 3520296 | 1.65 | 173642 |
| 33 | 23.221 | 22630392 | 10.58 | 235431 |
| 34 | 23.623 | 17865849 | 8.35 | 1114913 |
| 35 | 24.057 | 4708441 | 2.20 | 290733 |
| 36 | 24.359 | 4086311 | 1.91 | 276895 |
| 37 | 24.661 | 35429569 | 16.57 | 1765464 |
| 38 | 26.639 | 30653977 | 14.33 | 480095 |
| 39 | 26.977 | 5710437 | 2.67 | 393204 |
| 40 | 27.595 | 12852254 | 6.01 | 398867 |
| 41 | 27.911 | 21792801 | 10.19 | 563202 |

TABLE 7
HPLC fingerprint of extract AV016BaSu(65)04(100).

| | Retention | | | |
|---|---|---|---|---|
| | Time | Area | % Area | Height |
| 1 | 1.241 | 10089 | 0.00 | 743 |
| 2 | 1.737 | 139563 | 0.04 | 29368 |
| 3 | 1.897 | 10508564 | 3.02 | 2120419 |
| 4 | 2.474 | 168462 | 0.05 | 20793 |
| 5 | 3.560 | 244245 | 0.07 | 13079 |
| 6 | 5.156 | 118208 | 0.03 | 5005 |
| 7 | 6.455 | 515048 | 0.15 | 12630 |
| 8 | 7.011 | 397795 | 0.11 | 16720 |
| 9 | 7.792 | 1203998 | 0.35 | 35331 |
| 10 | 8.408 | 1782066 | 0.51 | 81114 |
| 11 | 9.287 | 3153097 | 0.90 | 85209 |
| 12 | 10.010 | 3363741 | 0.97 | 117298 |
| 13 | 10.521 | 3211695 | 0.92 | 129563 |
| 14 | 10.919 | 3272829 | 0.94 | 151739 |
| 15 | 11.201 | 2910895 | 0.84 | 187760 |
| 16 | 11.469 | 2011099 | 0.58 | 187543 |
| 17 | 12.570 | 22668982 | 6.51 | 608697 |
| 18 | 13.072 | 22315645 | 6.40 | 1372355 |
| 19 | 13.943 | 15908037 | 4.56 | 466306 |
| 20 | 14.267 | 13002097 | 3.73 | 755966 |
| 21 | 14.499 | 14043370 | 4.03 | 677681 |
| 22 | 15.168 | 18500419 | 5.31 | 803270 |
| 23 | 15.443 | 8340109 | 2.39 | 561189 |
| 24 | 15.645 | 7732494 | 2.22 | 554586 |
| 25 | 15.999 | 15671975 | 4.50 | 569336 |
| 26 | 16.333 | 14724201 | 4.23 | 556235 |
| 27 | 16.825 | 11690960 | 3.35 | 545534 |
| 28 | 17.178 | 11285355 | 3.24 | 522522 |
| 29 | 18.532 | 79310513 | 22.76 | 612406 |
| 30 | 19.864 | 57401105 | 16.47 | 441406 |
| 31 | 25.389 | 2531990 | 0.73 | 47750 |
| 32 | 29.165 | 4724 | 0.00 | 733 |
| 33 | 29.850 | 337961 | 0.10 | 60806 |

TABLE 8
HPLC fingerprint of extract AV016BaSu(65)06(100).

| | Retention | | | |
|---|---|---|---|---|
| | Time | Area | % Area | Height |
| 1 | 1.726 | 325346 | 0.17 | 47632 |
| 2 | 1.893 | 23371224 | 12.48 | 3640871 |
| 3 | 2.482 | 714853 | 0.38 | 60900 |
| 4 | 3.177 | 1875237 | 1.00 | 99118 |
| 5 | 3.690 | 485133 | 0.26 | 25416 |
| 6 | 4.103 | 679406 | 0.36 | 27133 |
| 7 | 4.359 | 352324 | 0.19 | 24825 |
| 8 | 4.857 | 272252 | 0.15 | 14769 |
| 9 | 5.596 | 1486044 | 0.79 | 49063 |
| 10 | 6.072 | 4857288 | 2.59 | 165611 |
| 11 | 7.074 | 715830 | 0.38 | 46771 |
| 12 | 7.247 | 928001 | 0.50 | 52209 |
| 13 | 7.925 | 1065970 | 0.57 | 45480 |
| 14 | 8.330 | 2132695 | 1.14 | 79394 |
| 15 | 9.096 | 2308356 | 1.23 | 80800 |
| 16 | 9.743 | 2396105 | 1.28 | 79390 |
| 17 | 11.179 | 12808928 | 6.84 | 205854 |
| 18 | 12.413 | 25125074 | 13.42 | 602504 |
| 19 | 13.130 | 1899754 | 1.01 | 160549 |
| 20 | 14.031 | 28439970 | 15.19 | 935325 |
| 21 | 14.646 | 9859006 | 5.27 | 326112 |
| 22 | 15.208 | 3351792 | 1.79 | 211034 |
| 23 | 15.744 | 11230538 | 6.00 | 540161 |
| 24 | 16.030 | 4547672 | 2.43 | 212887 |
| 25 | 16.651 | 5194350 | 2.77 | 232212 |
| 26 | 16.896 | 3162049 | 1.69 | 216298 |
| 27 | 17.244 | 5839204 | 3.12 | 208158 |
| 28 | 17.782 | 12488631 | 6.67 | 281615 |
| 29 | 18.729 | 4904610 | 2.62 | 146569 |
| 30 | 19.386 | 2196346 | 1.17 | 115299 |
| 31 | 19.673 | 8273125 | 4.42 | 99688 |
| 32 | 21.529 | 667601 | 0.36 | 50722 |
| 33 | 21.763 | 1946885 | 1.04 | 46660 |
| 34 | 22.802 | 1281629 | 0.68 | 26705 |
| 35 | 26.305 | 16760 | 0.01 | 1806 |
| 36 | 27.757 | 52103 | 0.03 | 5397 |

TABLE 9
HPLC fingerprint of extract AV016BaSu(105)08(100).

| | Retention | | | |
|---|---|---|---|---|
| | Time | Area | % Area | Height |
| 1 | 2.531 | 251954 | 2.83 | 45170 |
| 2 | 3.153 | 91508 | 1.03 | 7618 |
| 3 | 3.590 | 53827 | 0.60 | 2846 |
| 4 | 5.809 | 95383 | 1.07 | 5176 |
| 5 | 6.237 | 84290 | 0.95 | 3408 |
| 6 | 7.377 | 237161 | 2.66 | 7025 |
| 7 | 7.781 | 208394 | 2.34 | 12462 |
| 8 | 7.968 | 105188 | 1.18 | 10471 |
| 9 | 8.523 | 386758 | 4.34 | 15438 |
| 10 | 8.922 | 255053 | 2.86 | 13182 |
| 11 | 9.164 | 103465 | 1.16 | 11779 |
| 12 | 9.356 | 146409 | 1.64 | 11519 |
| 13 | 9.690 | 263555 | 2.96 | 11664 |
| 14 | 10.035 | 197458 | 2.22 | 12951 |
| 15 | 10.220 | 188840 | 2.12 | 14479 |
| 16 | 10.471 | 188182 | 2.11 | 14258 |
| 17 | 10.814 | 371997 | 4.18 | 13832 |
| 18 | 11.142 | 112966 | 1.27 | 12605 |
| 19 | 11.344 | 318863 | 3.58 | 13928 |
| 20 | 11.735 | 280148 | 3.15 | 11849 |
| 21 | 12.451 | 609274 | 6.84 | 21533 |
| 22 | 13.107 | 585271 | 6.57 | 38319 |
| 23 | 13.648 | 210651 | 2.37 | 8967 |
| 24 | 14.205 | 227248 | 2.55 | 13908 |
| 25 | 14.513 | 2594830 | 29.14 | 208042 |
| 26 | 15.951 | 40742 | 0.46 | 3155 |
| 27 | 16.683 | 52708 | 0.59 | 5023 |
| 28 | 17.691 | 70180 | 0.79 | 6181 |
| 29 | 24.062 | 198328 | 2.23 | 4386 |
| 30 | 24.791 | 13467 | 0.15 | 1398 |
| 31 | 24.993 | 12465 | 0.14 | 996 |
| 32 | 25.557 | 2542 | 0.03 | 494 |
| 33 | 26.037 | 62889 | 0.71 | 3648 |
| 34 | 26.578 | 50279 | 0.56 | 5059 |
| 35 | 28.119 | 5587 | 0.06 | 665 |
| 36 | 28.951 | 42494 | 0.48 | 3689 |
| 37 | 29.707 | 184276 | 2.07 | 17288 |

TABLE 10
HPLC fingerprint of extract AV016Fr(105)08(100).

| | Retention | | | |
|---|---|---|---|---|
| | Time | Area | % Area | Height |
| 1 | 2.238 | 10762330 | 4.73 | 1150702 |
| 2 | 2.553 | 5628824 | 2.47 | 655829 |
| 3 | 2.914 | 2203056 | 0.97 | 125091 |
| 4 | 3.208 | 3512641 | 1.54 | 318280 |
| 5 | 3.357 | 1070868 | 0.47 | 212573 |
| 6 | 3.602 | 1899749 | 0.84 | 121669 |
| 7 | 4.052 | 3407226 | 1.50 | 136020 |
| 8 | 4.503 | 2919013 | 1.28 | 94633 |
| 9 | 5.093 | 2190949 | 0.96 | 89619 |
| 10 | 5.889 | 12371237 | 5.44 | 338787 |
| 11 | 6.223 | 10361831 | 4.55 | 315486 |
| 12 | 7.163 | 4782640 | 2.10 | 237340 |
| 13 | 7.700 | 40604091 | 17.85 | 1251589 |
| 14 | 9.163 | 11851832 | 5.21 | 268920 |
| 15 | 9.857 | 3502688 | 1.54 | 228636 |
| 16 | 10.125 | 4862586 | 2.14 | 227948 |
| 17 | 10.448 | 2710010 | 1.19 | 231182 |
| 18 | 10.625 | 2889073 | 1.27 | 233695 |
| 19 | 10.818 | 1656694 | 0.73 | 207765 |
| 20 | 11.218 | 23908811 | 10.51 | 1250520 |
| 21 | 12.067 | 40804495 | 17.94 | 1657258 |
| 22 | 13.556 | 507546 | 0.22 | 127495 |
| 23 | 13.790 | 6006453 | 2.64 | 134107 |
| 24 | 14.940 | 8534623 | 3.75 | 102587 |
| 25 | 17.771 | 960263 | 0.42 | 27876 |
| 26 | 18.313 | 820905 | 0.36 | 25276 |
| 27 | 20.696 | 13722 | 0.01 | 1129 |
| 28 | 23.988 | 77588 | 0.03 | 2021 |
| 29 | 24.355 | 9612 | 0.00 | 1658 |
| 30 | 25.622 | 204766 | 0.09 | 9215 |
| 31 | 25.787 | 301167 | 0.13 | 11470 |
| 32 | 26.472 | 205806 | 0.09 | 9613 |
| 33 | 27.231 | 486773 | 0.21 | 12154 |
| 34 | 28.270 | 5013669 | 2.20 | 94934 |
| 35 | 29.001 | 2956931 | 1.30 | 213028 |
| 36 | 29.400 | 7494149 | 3.29 | 238402 |

TABLE 11
LC/MS Fingerprint of extract AV016BaDi(28)04(20)
(TIC Spectrum (Q1 +ve mode)
Peak List:

| Time | Area | | Height | | Width | |
|---|---|---|---|---|---|---|
| (min) | (counts) | % Area | (cps) | % Height | (min) | Baseline Type |
| 2.1775 | 1.06E+08 | 12.3131 | 1.97E+07 | 17.7412 | 0.1874 | Valley |
| 2.3167 | 3.07E+08 | 35.522 | 3.45E+07 | 31.1894 | 0.2408 | Valley |
| 2.8588 | 2.07E+07 | 2.398 | 2.52E+06 | 2.2768 | 0.2408 | Base to Base |
| 3.2879 | 1.84E+07 | 2.1314 | 2.99E+06 | 2.7 | 0.1873 | Base to Base |
| 3.7661 | 1.19E+07 | 1.3822 | 1.74E+06 | 1.5668 | 0.2408 | Base to Base |
| 5.0086 | 9.58E+06 | 1.1091 | 1.08E+06 | 0.9759 | 0.2941 | Base to Base |
| 6.1538 | 8.09E+06 | 0.9365 | 1.03E+06 | 0.9294 | 0.2141 | Base to Base |
| 6.8962 | 6.00E+06 | 0.6949 | 1.39E+06 | 1.2572 | 0.1605 | Base to Base |
| 7.7876 | 8.31E+06 | 0.9627 | 1.05E+06 | 0.9452 | 0.2676 | Valley |
| 7.9719 | 1.18E+07 | 1.3624 | 1.89E+06 | 1.7057 | 0.214 | Valley |
| 10.735 | 8.06E+06 | 0.9334 | 1.45E+06 | 1.3135 | 0.2408 | Base to Base |
| 12.0425 | 2.48E+06 | 0.2866 | 6.74E+05 | 0.6081 | 0.107 | Base to Base |
| 12.3092 | 1.54E+06 | 0.1783 | 8.28E+05 | 0.7472 | 0.0803 | Base to Base |
| 12.7311 | 4.83E+06 | 0.5597 | 9.68E+05 | 0.8739 | 0.1605 | Base to Base |
| 14.3893 | 3.76E+06 | 0.4351 | 9.54E+05 | 0.8611 | 0.1605 | Base to Base |
| 15.0886 | 3.66E+06 | 0.4237 | 9.12E+05 | 0.8235 | 0.1338 | Base to Base |
| 17.5521 | 1.82E+07 | 2.1125 | 2.34E+06 | 2.1145 | 0.3211 | Base to Base |
| 17.9476 | 1.90E+07 | 2.1971 | 1.86E+06 | 1.6753 | 0.2408 | Base to Base |
| 19.5586 | 1.44E+07 | 1.6648 | 1.53E+06 | 1.3819 | 0.3211 | Base to Base |
| 20.3886 | 4.78E+06 | 0.553 | 1.22E+06 | 1.1028 | 0.1605 | Base to Base |
| 21.5431 | 1.49E+07 | 1.7195 | 1.31E+06 | 1.1854 | 0.3746 | Base to Base |
| 23.6233 | 8.28E+06 | 0.9586 | 1.77E+06 | 1.5935 | 0.1873 | Base to Base |
| 24.5583 | 8.58E+06 | 0.9939 | 1.18E+06 | 1.0651 | 0.2676 | Base to Base |
| 25.1938 | 9.77E+06 | 1.131 | 1.24E+06 | 1.117 | 0.3211 | Base to Base |
| 25.6005 | 5.11E+07 | 5.9143 | 5.26E+06 | 4.7467 | 0.3478 | Base to Base |
| 25.9137 | 1.34E+07 | 1.5528 | 1.76E+06 | 1.5921 | 0.2141 | Base to Base |
| 26.7469 | 1.82E+07 | 2.1106 | 2.05E+06 | 1.8533 | 0.2943 | Base to Base |
| 27.3985 | 1.09E+07 | 1.2594 | 1.39E+06 | 1.2536 | 0.2675 | Base to Base |
| 27.6349 | 4.60E+06 | 0.5329 | 1.06E+06 | 0.9601 | 0.1338 | Base to Base |
| 28.7272 | 1.89E+07 | 2.1893 | 1.77E+06 | 1.5968 | 0.3478 | Base to Base |
| 28.8934 | 1.03E+07 | 1.1905 | 2.09E+06 | 1.8883 | 0.1338 | Base to Base |
| 29.1613 | 3.01E+06 | 0.3489 | 9.23E+05 | 0.8329 | 0.1338 | Base to Base |
| 29.9922 | 8.20E+07 | 9.495 | 5.03E+06 | 4.5397 | 0.6957 | Base to Base |
| 34.628 | 1.16E+07 | 1.3485 | 1.01E+06 | 0.9137 | 0.3746 | Base to Base |
| 36.8649 | 2.86E+06 | 0.3315 | 8.91E+05 | 0.804 | 0.107 | Base to Base |
| 37.1874 | 1.77E+06 | 0.2051 | 6.81E+05 | 0.6146 | 0.0802 | Base to Base |
| 37.7494 | 4.85E+06 | 0.5616 | 7.24E+05 | 0.6535 | 0.1873 | Base to Base |

TABLE 12
LC/MS Fingerprint of extract AV016BaDi(28)04(20)
(TIC Spectrum (Q1 −ve mode)
Peak List:

| Time | Area | | Height | | Width | |
|---|---|---|---|---|---|---|
| (min) | (counts) | % Area | (cps) | % Height | (min) | Baseline Type |
| 2.1961 | 2.85E+08 | 24.4168 | 3.04E+07 | 25.3851 | 0.2676 | Valley |
| 2.4834 | 4.77E+08 | 40.8241 | 2.58E+07 | 21.5134 | 0.5084 | Valley |
| 3.4214 | 7.47E+06 | 0.6387 | 1.44E+06 | 1.2067 | 0.1873 | Base to Base |
| 4.4188 | 3.03E+06 | 0.2592 | 1.25E+06 | 1.0442 | 0.107 | Base to Base |
| 7.9809 | 8.39E+06 | 0.7181 | 1.33E+06 | 1.1079 | 0.1873 | Base to Base |
| 9.6559 | 5.75E+06 | 0.4914 | 1.14E+06 | 0.9551 | 0.1873 | Base to Base |
| 11.4246 | 2.60E+06 | 0.2223 | 8.53E+05 | 0.7128 | 0.1338 | Base to Base |
| 12.387 | 6.72E+06 | 0.575 | 1.29E+06 | 1.0785 | 0.1873 | Base to Base |
| 12.7886 | 3.28E+07 | 2.8027 | 4.15E+06 | 3.4643 | 0.2408 | Base to Base |
| 14.0996 | 3.48E+06 | 0.2974 | 1.12E+06 | 0.9338 | 0.107 | Base to Base |
| 14.8736 | 3.87E+06 | 0.3307 | 5.13E+05 | 0.4282 | 0.1605 | Base to Base |
| 15.1974 | 2.00E+06 | 0.1714 | 1.08E+06 | 0.9018 | 0.0803 | Base to Base |
| 16.2118 | 1.20E+07 | 1.0275 | 1.28E+06 | 1.0691 | 0.2676 | Base to Base |
| 17.1494 | 6.42E+06 | 0.5495 | 1.26E+06 | 1.0499 | 0.214 | Base to Base |
| 17.3337 | 1.35E+07 | 1.157 | 2.57E+06 | 2.1439 | 0.1873 | Base to Base |
| 17.6906 | 3.73E+07 | 3.1891 | 2.51E+06 | 2.0939 | 0.4013 | Valley |
| 17.8922 | 9.86E+06 | 0.8435 | 1.99E+06 | 1.6607 | 0.1338 | Valley |
| 18.3572 | 4.74E+06 | 0.4051 | 2.29E+06 | 1.9131 | 0.0803 | Base to Base |
| 18.7319 | 9.90E+06 | 0.8468 | 1.37E+06 | 1.144 | 0.2408 | Base to Base |
| 19.6901 | 6.66E+06 | 0.5693 | 1.68E+06 | 1.4049 | 0.1338 | Base to Base |
| 20.1151 | 1.26E+07 | 1.082 | 1.77E+06 | 1.4749 | 0.2408 | Base to Base |
| 21.084 | 5.17E+06 | 0.4419 | 1.45E+06 | 1.2082 | 0.1338 | Base to Base |
| 21.4301 | 6.15E+06 | 0.5263 | 2.11E+06 | 1.7665 | 0.0803 | Valley |
| 21.5887 | 1.05E+07 | 0.8972 | 2.53E+06 | 2.1134 | 0.1605 | Valley |
| 23.7467 | 8.39E+06 | 0.7173 | 1.24E+06 | 1.0376 | 0.2675 | Base to Base |
| 23.9916 | 4.44E+06 | 0.3795 | 8.19E+05 | 0.6845 | 0.1338 | Base to Base |
| 24.7774 | 6.82E+06 | 0.5835 | 1.14E+06 | 0.9516 | 0.2943 | Base to Base |
| 25.5831 | 2.43E+07 | 2.0804 | 2.38E+06 | 1.9909 | 0.3211 | Base to Base |
| 25.9059 | 1.37E+07 | 1.1725 | 2.04E+06 | 1.7081 | 0.2675 | Base to Base |
| 26.2502 | 1.00E+07 | 0.8555 | 1.08E+06 | 0.9016 | 0.2408 | Base to Base |
| 26.7168 | 1.72E+07 | 1.471 | 2.26E+06 | 1.8859 | 0.2408 | Valley |
| 26.9142 | 2.14E+07 | 1.8288 | 2.20E+06 | 1.8395 | 0.2676 | Valley |
| 28.3515 | 3.10E+07 | 2.6494 | 3.47E+06 | 2.8963 | 0.4281 | Valley |
| 28.6747 | 2.53E+07 | 2.1615 | 3.53E+06 | 2.9518 | 0.3478 | Valley |
| 28.9455 | 4.47E+06 | 0.3823 | 1.21E+06 | 1.0119 | 0.1338 | Base to Base |
| 29.6212 | 7.70E+06 | 0.6586 | 7.87E+05 | 0.6574 | 0.2408 | Base to Base |
| 30.0197 | 1.10E+07 | 0.939 | 2.58E+06 | 2.1565 | 0.1873 | Base to Base |
| 30.4766 | 9.79E+06 | 0.8377 | 1.86E+06 | 1.552 | 0.1605 | Base to Base |

TABLE 13
LC/MS Fingerprint of extract AV016BaDi(65)04(100)
(TIC Spectrum (Q1 +ve mode)
Peak List:

| Time | Area | | Height | | Width | |
|---|---|---|---|---|---|---|
| (min) | (counts) | % Area | (cps) | % Height | (min) | Baseline Type |
| 1.6819 | 5.13E+07 | 3.3673 | 1.07E+07 | 7.702 | 0.1873 | Base to Base |
| 2.1223 | 4.05E+07 | 2.6634 | 4.33E+06 | 3.1211 | 0.2408 | Base to Base |
| 2.4137 | 3.19E+08 | 20.9505 | 4.49E+07 | 32.3621 | 0.3479 | Base to Base |
| 35.37 | 7.19E+07 | 4.7236 | 3.26E+06 | 2.3544 | 0.6422 | Base to Base |
| 43.1584 | 2.30E+08 | 15.0758 | 1.02E+07 | 7.3293 | 0.7759 | Base to Base |
| 46.4236 | 1.38E+07 | 0.9069 | 1.66E+06 | 1.1971 | 0.3746 | Base to Base |
| 48.1523 | 2.43E+07 | 1.5953 | 2.55E+06 | 1.8369 | 0.3211 | Base to Base |
| 48.5731 | 7.28E+07 | 4.7831 | 6.02E+06 | 4.3446 | 0.3746 | Base to Base |
| 49.1844 | 4.59E+07 | 3.0145 | 4.22E+06 | 3.0466 | 0.4014 | Base to Base |
| 49.7221 | 1.53E+08 | 10.0401 | 1.05E+07 | 7.5644 | 0.6421 | Base to Base |
| 50.41 | 1.98E+08 | 13.0047 | 1.31E+07 | 9.4266 | 0.6422 | Base to Base |
| 53.0591 | 1.83E+07 | 1.2049 | 2.30E+06 | 1.6573 | 0.3478 | Base to Base |
| 53.7262 | 1.26E+08 | 8.2723 | 8.93E+06 | 6.4387 | 0.5619 | Base to Base |
| 55.4126 | 1.19E+08 | 7.8049 | 1.12E+07 | 8.068 | 0.4013 | Base to Base |
| 55.8032 | 3.95E+07 | 2.5926 | 4.92E+06 | 3.5508 | 0.3478 | Base to Base |

TABLE 14
LC/MS Fingerprint of extract AV016BaDi(65)04(100)
(TIC Spectrum (Q1 −ve mode)
Peak List:

| Time | Area | | Height | | Width | |
|---|---|---|---|---|---|---|
| (min) | (counts) | % Area | (cps) | % Height | (min) | Baseline Type |
| 1.5858 | 4.95E+08 | 33.2934 | 5.09E+07 | 33.5845 | 0.3211 | Valley |
| 1.6837 | 4.68E+08 | 31.4658 | 4.70E+07 | 30.9815 | 0.2675 | Valley |
| 2.281 | 2.71E+08 | 18.2632 | 2.23E+07 | 14.6901 | 0.3479 | Valley |
| 2.3736 | 1.40E+08 | 9.4462 | 2.07E+07 | 13.6345 | 0.214 | Valley |
| 42.7019 | 3.33E+06 | 0.2239 | 1.08E+06 | 0.7156 | 0.2141 | Base to Base |
| 51.6555 | 9.78E+06 | 0.6583 | 1.30E+06 | 0.8605 | 0.2408 | Base to Base |
| 54.1648 | 7.99E+07 | 5.3782 | 4.28E+06 | 2.8245 | 0.7224 | Base to Base |
| 55.1059 | 3.72E+06 | 0.2501 | 1.06E+06 | 0.697 | 0.107 | Base to Base |
| 57.6108 | 6.57E+06 | 0.4422 | 1.30E+06 | 0.855 | 0.214 | Base to Base |
| 58.0334 | 6.71E+06 | 0.4517 | 9.03E+05 | 0.5959 | 0.2141 | Base to Base |
| 58.9956 | 1.89E+06 | 0.127 | 8.50E+05 | 0.5609 | 0.0802 | Base to Base |

TABLE 15
LC/MS Fingerprint of extract AV016BaSu(65)09(100)
(TIC Spectrum (Q1 +ve mode).
Peak List

| Time | Area | | Height | | Width | |
|---|---|---|---|---|---|---|
| (min) | (counts) | % Area | (cps) | % Height | (min) | Baseline Type |
| 0.6837 | 2.30E+07 | 1.9654 | 2.66E+06 | 2.8378 | 0.3211 | Base to Base |
| 1.7359 | 3.23E+08 | 27.6122 | 2.10E+07 | 22.4515 | 0.6155 | Base to Base |
| 2.1817 | 6.42E+08 | 54.9509 | 5.24E+07 | 55.9679 | 0.3212 | Base to Base |
| 46.2873 | 2.68E+06 | 0.2294 | 1.06E+06 | 1.1283 | 0.0802 | Base to Base |
| 51.3977 | 1.34E+07 | 1.1443 | 1.86E+06 | 1.987 | 0.2408 | Base to Base |
| 51.7551 | 1.14E+07 | 0.9773 | 2.30E+06 | 2.4614 | 0.1605 | Base to Base |
| 53.5882 | 4.82E+07 | 4.1246 | 4.09E+06 | 4.3665 | 0.4281 | Base to Base |
| 56.8025 | 3.06E+07 | 2.6205 | 2.29E+06 | 2.4491 | 0.2943 | Base to Base |
| 57.7937 | 7.45E+07 | 6.3753 | 5.94E+06 | 6.3504 | 0.4549 | Base to Base |

TABLE 16
LC/MS Fingerprint of extract AV016BaSu(65)01(100)
(TIC Spectrum (Q1 +ve mode)
Peak List:

| Time | Area | | Height | | Width | |
|---|---|---|---|---|---|---|
| (min) | (counts) | % Area | (cps) | % Height | (min) | Baseline Type |
| 0.9306 | 6.79E+06 | 0.9084 | 1.15E+06 | 1.8547 | 0.1873 | Base to Base |
| 1.6126 | 3.36E+07 | 4.493 | 1.23E+06 | 1.9756 | 0.2943 | Base to Base |
| 2.2467 | 5.48E+08 | 73.275 | 2.92E+07 | 46.9161 | 0.5887 | Base to Base |
| 2.6959 | 5.61E+06 | 0.7509 | 2.37E+06 | 3.8115 | 0.0803 | Base to Base |
| 8.7292 | 4.57E+06 | 0.6115 | 7.87E+05 | 1.2645 | 0.1605 | Base to Base |
| 22.1814 | 1.19E+07 | 1.5974 | 9.94E+05 | 1.5977 | 0.3478 | Base to Base |
| 22.538 | 5.22E+06 | 0.6979 | 8.29E+05 | 1.3331 | 0.1873 | Base to Base |
| 24.7315 | 5.89E+06 | 0.7887 | 1.07E+06 | 1.7263 | 0.1605 | Base to Base |
| 26.7098 | 3.29E+06 | 0.4408 | 1.01E+06 | 1.6234 | 0.107 | Base to Base |
| 28.9401 | 1.24E+07 | 1.6572 | 1.40E+06 | 2.2533 | 0.2676 | Base to Base |
| 29.316 | 3.87E+06 | 0.5185 | 1.47E+06 | 2.3673 | 0.0803 | Base to Base |
| 33.8775 | 4.43E+06 | 0.5927 | 1.17E+06 | 1.883 | 0.1605 | Base to Base |
| 35.7436 | 4.34E+06 | 0.5805 | 8.92E+05 | 1.4329 | 0.1873 | Base to Base |
| 37.7239 | 6.26E+06 | 0.8374 | 1.17E+06 | 1.8777 | 0.214 | Base to Base |
| 37.8343 | 1.99E+06 | 0.2659 | 7.72E+05 | 1.2402 | 0.107 | Base to Base |
| 39.1183 | 5.39E+06 | 0.7212 | 9.96E+05 | 1.6006 | 0.1606 | Base to Base |
| 44.5179 | 7.33E+06 | 0.9815 | 9.63E+05 | 1.5473 | 0.2676 | Base to Base |
| 49.8635 | 3.66E+06 | 0.4897 | 8.38E+05 | 1.347 | 0.1338 | Base to Base |
| 51.9325 | 3.25E+06 | 0.4342 | 1.23E+06 | 1.9711 | 0.0803 | Base to Base |
| 52.2278 | 3.39E+06 | 0.4532 | 1.25E+06 | 2.0023 | 0.1071 | Base to Base |
| 52.5806 | 1.33E+07 | 1.7735 | 1.73E+06 | 2.7832 | 0.2676 | Valley |
| 52.735 | 1.12E+07 | 1.5033 | 1.69E+06 | 2.7234 | 0.1873 | Valley |
| 53.8915 | 1.27E+07 | 1.706 | 2.22E+06 | 3.5732 | 0.2141 | Base to Base |
| 54.4726 | 7.86E+06 | 1.0524 | 1.51E+06 | 2.4278 | 0.1338 | Base to Base |
| 57.8304 | 1.22E+07 | 1.6307 | 2.45E+06 | 3.9445 | 0.1873 | Base to Base |
| 59.5907 | 5.79E+06 | 0.7754 | 1.11E+06 | 1.7919 | 0.1338 | Base to Base |
| 59.9224 | 3.46E+06 | 0.463 | 7.03E+05 | 1.1303 | 0.1338 | Base to Base |

TABLE 17
LC/MS Fingerprint of extract AV016BaSu(65)01(100)
(TIC Spectrum (Q1 −ve mode)

| Time | Area | | Height | | Width | |
|---|---|---|---|---|---|---|
| (min) | (counts) | % Area | (cps) | % Height | (min) | Baseline Type |
| 0.9354 | 4.30E+06 | 1.2293 | 1.22E+06 | 2.4333 | 0.214 | Base to Base |
| 2.2806 | 1.29E+08 | 36.7178 | 1.08E+07 | 21.6409 | 0.4549 | Base to Base |
| 2.6726 | 4.11E+07 | 11.7354 | 1.62E+07 | 32.297 | 0.0803 | Base to Base |
| 2.9712 | 3.79E+07 | 10.8273 | 7.74E+06 | 15.4562 | 0.1873 | Base to Base |
| 51.3175 | 1.61E+06 | 0.4586 | 6.88E+05 | 1.3736 | 0.0803 | Base to Base |
| 53.8048 | 2.24E+07 | 6.3996 | 1.62E+06 | 3.2379 | 0.3211 | Base to Base |
| 54.3764 | 3.18E+07 | 9.0759 | 2.29E+06 | 4.5779 | 0.5351 | Base to Base |
| 56.2363 | 1.47E+07 | 4.2035 | 1.55E+06 | 3.1017 | 0.3478 | Base to Base |
| 56.9396 | 2.67E+06 | 0.7637 | 1.65E+06 | 3.2962 | 0.0535 | Base to Base |
| 57.4693 | 1.54E+07 | 4.4064 | 2.12E+06 | 4.2359 | 0.2676 | Valley |
| 57.8919 | 4.50E+07 | 12.8633 | 3.30E+06 | 6.595 | 0.5886 | Valley |
| 58.5708 | 4.62E+06 | 1.3192 | 8.79E+05 | 1.7544 | 0.2141 | Base to Base |

TABLE 18
LC/MS Fingerprint of extract AV016BaSu(65)01(100)ng
(TIC Spectrum (Q1 +ve mode)

| Time | Area | | Height | | Width | |
|---|---|---|---|---|---|---|
| (min) | (counts) | % Area | (cps) | % Height | (min) | Baseline Type |
| 0.2815 | 2.64E+07 | 2.56 | 1.89E+06 | 2.0165 | 0.4014 | Base to Base |
| 0.6177 | 2.15E+07 | 2.0898 | 4.89E+06 | 5.2322 | 0.214 | Base to Base |
| 1.4595 | 6.17E+07 | 5.9816 | 5.53E+06 | 5.911 | 0.3478 | Valley |
| 1.6305 | 2.63E+07 | 2.5549 | 3.33E+06 | 3.5575 | 0.1873 | Valley |
| 2.2215 | 5.21E+08 | 50.5534 | 3.02E+07 | 32.3009 | 0.5352 | Base to Base |
| 4.4158 | 5.86E+06 | 0.5685 | 1.43E+06 | 1.5317 | 0.1605 | Base to Base |
| 10.5664 | 6.14E+06 | 0.5957 | 7.92E+05 | 0.8469 | 0.2408 | Base to Base |
| 23.1719 | 7.20E+06 | 0.6985 | 1.10E+06 | 1.1733 | 0.2141 | Base to Base |
| 26.6471 | 2.14E+06 | 0.2071 | 1.07E+06 | 1.1409 | 0.0803 | Base to Base |
| 28.4723 | 8.22E+06 | 0.7973 | 9.73E+05 | 1.04 | 0.2676 | Base to Base |
| 29.1292 | 3.99E+06 | 0.387 | 7.08E+05 | 0.7574 | 0.1873 | Base to Base |
| 31.5736 | 8.16E+06 | 0.7917 | 8.24E+05 | 0.8813 | 0.2408 | Base to Base |
| 31.6945 | 2.56E+06 | 0.2481 | 7.61E+05 | 0.8141 | 0.107 | Base to Base |
| 33.9826 | 1.00E+07 | 0.9715 | 1.28E+06 | 1.3722 | 0.2943 | Base to Base |
| 36.5048 | 4.65E+06 | 0.4512 | 8.70E+05 | 0.93 | 0.1338 | Base to Base |
| 37.6212 | 3.23E+06 | 0.3131 | 1.19E+06 | 1.2726 | 0.107 | Base to Base |
| 39.8509 | 3.18E+06 | 0.3083 | 7.69E+05 | 0.822 | 0.1338 | Base to Base |
| 42.0646 | 4.16E+06 | 0.4038 | 9.70E+05 | 1.0375 | 0.1605 | Base to Base |
| 42.4115 | 3.05E+06 | 0.2954 | 6.68E+05 | 0.7142 | 0.1338 | Base to Base |
| 42.5886 | 5.36E+06 | 0.5203 | 1.14E+06 | 1.2137 | 0.1338 | Base to Base |
| 43.7214 | 1.66E+06 | 0.1606 | 8.64E+05 | 0.9238 | 0.0803 | Base to Base |
| 49.6266 | 3.44E+06 | 0.3332 | 6.23E+05 | 0.6663 | 0.1605 | Base to Base |
| 49.8477 | 1.80E+06 | 0.175 | 9.30E+05 | 0.9949 | 0.0803 | Base to Base |
| 50.2725 | 2.67E+06 | 0.2585 | 1.22E+06 | 1.3044 | 0.0803 | Base to Base |
| 50.4625 | 2.57E+06 | 0.2489 | 1.25E+06 | 1.3321 | 0.0803 | Base to Base |
| 51.6971 | 9.21E+06 | 0.8936 | 1.33E+06 | 1.4175 | 0.3211 | Base to Base |
| 52.2644 | 3.77E+06 | 0.3661 | 1.08E+06 | 1.1526 | 0.1338 | Base to Base |
| 52.6743 | 1.05E+07 | 1.0181 | 1.27E+06 | 1.3617 | 0.3478 | Base to Base |
| 53.3229 | 1.80E+06 | 0.175 | 7.11E+05 | 0.7607 | 0.0803 | Base to Base |
| 54.0466 | 5.16E+07 | 5.0087 | 5.02E+06 | 5.3685 | 0.4281 | Base to Base |
| 54.5611 | 7.42E+06 | 0.7194 | 1.35E+06 | 1.439 | 0.1873 | Base to Base |
| 55.786 | 3.29E+06 | 0.3187 | 1.22E+06 | 1.304 | 0.107 | Base to Base |
| 56.3205 | 9.29E+06 | 0.9008 | 1.81E+06 | 1.9397 | 0.1605 | Base to Base |
| 56.982 | 1.01E+07 | 0.9748 | 1.91E+06 | 2.0474 | 0.1873 | Base to Base |
| 57.5849 | 6.16E+07 | 5.9742 | 5.18E+06 | 5.5408 | 0.4549 | Valley |
| 57.9722 | 1.07E+08 | 10.4 | 6.43E+06 | 6.8743 | 0.6421 | Valley |
| 59.2956 | 8.01E+06 | 0.7772 | 9.41E+05 | 1.0067 | 0.2676 | Base to Base |

TABLE 19
LC/MS Fingerprint of extract AV016BaSu(65)01(100)g
(TIC Spectrum (Q1 +ve mode)

| Time | Area | | Height | | Width | |
|---|---|---|---|---|---|---|
| (min) | (counts) | % Area | (cps) | % Height | (min) | Baseline Type |
| 0.2462 | 4.54E+06 | 0.4516 | 1.28E+06 | 1.5367 | 0.1605 | Base to Base |
| 0.7783 | 2.91E+07 | 2.896 | 2.30E+06 | 2.7508 | 0.4281 | Base to Base |
| 1.4229 | 1.44E+08 | 14.3325 | 1.54E+07 | 18.4396 | 0.2676 | Valley |
| 1.6808 | 2.48E+08 | 24.6093 | 1.67E+07 | 20.0141 | 0.4281 | Valley |
| 2.2103 | 5.05E+08 | 50.1865 | 3.12E+07 | 37.4091 | 0.5084 | Base to Base |
| 2.6741 | 2.06E+07 | 2.0484 | 6.02E+06 | 7.2093 | 0.1338 | Base to Base |
| 3.5511 | 4.01E+06 | 0.3982 | 1.49E+06 | 1.7817 | 0.107 | Base to Base |
| 4.8158 | 1.41E+07 | 1.4004 | 2.22E+06 | 2.6599 | 0.2676 | Base to Base |
| 27.5026 | 4.41E+06 | 0.4385 | 1.10E+06 | 1.3192 | 0.2141 | Base to Base |
| 53.9115 | 1.52E+07 | 1.5124 | 1.73E+06 | 2.0716 | 0.3479 | Base to Base |
| 55.1432 | 1.04E+07 | 1.0317 | 1.45E+06 | 1.735 | 0.3478 | Base to Base |
| 56.3186 | 2.19E+06 | 0.2172 | 8.58E+05 | 1.0289 | 0.0803 | Base to Base |
| 57.4751 | 1.54E+06 | 0.153 | 7.41E+05 | 0.8876 | 0.0803 | Base to Base |
| 57.9589 | 3.26E+06 | 0.3241 | 9.65E+05 | 1.1566 | 0.107 | Base to Base |

TABLE 20
LC/MS Fingerprint of extract AV016BaSu(65)04(100)
(TIC Spectrum (Q1 +ve mode)

| Time | Area | | Height | | Width | |
|---|---|---|---|---|---|---|
| (min) | (counts) | % Area | (cps) | % Height | (min) | Baseline Type |
| 1.3402 | 1.14E+08 | 17.1348 | 1.35E+07 | 13.7133 | 0.2676 | Valley |
| 1.6073 | 1.74E+07 | 2.6038 | 1.93E+06 | 1.9612 | 0.1606 | Valley |
| 2.2967 | 1.87E+08 | 28.0404 | 1.01E+07 | 10.3069 | 0.4817 | Base to Base |
| 4.0358 | 4.53E+06 | 0.6786 | 1.01E+06 | 1.0332 | 0.1338 | Valley |
| 4.1691 | 3.10E+06 | 0.4647 | 1.31E+06 | 1.3305 | 0.0803 | Valley |
| 4.385 | 5.02E+06 | 0.7515 | 1.42E+06 | 1.4467 | 0.1338 | Base to Base |
| 4.9989 | 5.94E+06 | 0.8892 | 1.16E+06 | 1.1771 | 0.1605 | Base to Base |
| 5.3019 | 3.46E+06 | 0.5183 | 1.06E+06 | 1.0776 | 0.1338 | Base to Base |
| 5.9113 | 2.78E+06 | 0.4167 | 1.30E+06 | 1.3198 | 0.0803 | Base to Base |
| 6.1778 | 4.49E+06 | 0.6721 | 7.58E+05 | 0.7719 | 0.1873 | Base to Base |
| 6.3704 | 2.71E+06 | 0.4056 | 7.84E+05 | 0.7985 | 0.107 | Base to Base |
| 6.8924 | 6.19E+06 | 0.9275 | 7.75E+05 | 0.7891 | 0.214 | Base to Base |
| 7.2702 | 3.77E+06 | 0.5654 | 1.17E+06 | 1.1885 | 0.107 | Base to Base |
| 7.4357 | 2.04E+06 | 0.3051 | 9.28E+05 | 0.945 | 0.0803 | Base to Base |
| 7.9721 | 4.93E+06 | 0.7391 | 9.22E+05 | 0.939 | 0.1873 | Base to Base |
| 8.8605 | 6.09E+06 | 0.9128 | 7.53E+05 | 0.7667 | 0.2141 | Base to Base |
| 9.4252 | 2.87E+06 | 0.4292 | 7.85E+05 | 0.7996 | 0.1071 | Base to Base |
| 9.789 | 4.10E+06 | 0.6138 | 6.12E+05 | 0.6234 | 0.2676 | Base to Base |
| 10.7492 | 1.94E+06 | 0.2902 | 7.58E+05 | 0.7718 | 0.0803 | Base to Base |
| 13.0858 | 2.60E+06 | 0.3897 | 6.98E+05 | 0.7105 | 0.1094 | Valley |
| 13.1888 | 3.36E+06 | 0.5029 | 7.11E+05 | 0.7238 | 0.1582 | Valley |
| 15.4704 | 4.27E+06 | 0.64 | 1.29E+06 | 1.3121 | 0.1338 | Base to Base |
| 16.5549 | 2.37E+06 | 0.3545 | 8.66E+05 | 0.8822 | 0.0802 | Base to Base |
| 17.1515 | 4.19E+06 | 0.6275 | 1.24E+06 | 1.2608 | 0.107 | Valley |
| 17.2577 | 5.17E+06 | 0.7752 | 9.38E+05 | 0.9557 | 0.1606 | Valley |
| 17.4256 | 5.27E+06 | 0.7899 | 8.05E+05 | 0.8196 | 0.1605 | Base to Base |
| 17.7689 | 5.45E+06 | 0.816 | 1.09E+06 | 1.1149 | 0.1338 | Base to Base |
| 19.3648 | 2.95E+06 | 0.4416 | 8.37E+05 | 0.8528 | 0.1606 | Base to Base |
| 19.5311 | 1.19E+06 | 0.1786 | 5.57E+05 | 0.5667 | 0.0803 | Base to Base |
| 20.302 | 5.74E+06 | 0.8605 | 9.81E+05 | 0.9987 | 0.1605 | Base to Base |
| 23.1422 | 4.84E+06 | 0.7254 | 8.61E+05 | 0.8767 | 0.1605 | Base to Base |
| 23.4055 | 3.51E+06 | 0.5262 | 7.76E+05 | 0.7901 | 0.1345 | Base to Base |
| 25.5012 | 2.20E+06 | 0.3299 | 1.04E+06 | 1.0621 | 0.0803 | Base to Base |
| 25.9266 | 2.59E+06 | 0.3882 | 9.14E+05 | 0.931 | 0.107 | Base to Base |
| 26.7339 | 3.31E+06 | 0.496 | 1.09E+06 | 1.1142 | 0.107 | Base to Base |
| 27.8535 | 3.91E+06 | 0.5853 | 1.02E+06 | 1.0382 | 0.1873 | Base to Base |
| 28.7851 | 2.65E+06 | 0.3968 | 9.95E+05 | 1.0129 | 0.0803 | Valley |
| 28.9276 | 5.31E+06 | 0.7949 | 8.69E+05 | 0.8849 | 0.1873 | Valley |
| 29.0503 | 4.46E+06 | 0.6684 | 1.13E+06 | 1.1496 | 0.1605 | Valley |
| 29.6159 | 2.76E+06 | 0.413 | 1.10E+06 | 1.1195 | 0.107 | Base to Base |
| 31.7858 | 2.66E+06 | 0.3987 | 7.40E+05 | 0.7538 | 0.1338 | Valley |
| 31.9243 | 2.84E+06 | 0.4248 | 5.33E+05 | 0.543 | 0.1605 | Valley |
| 32.5063 | 3.51E+06 | 0.5259 | 1.20E+06 | 1.2204 | 0.107 | Base to Base |
| 33.128 | 3.73E+06 | 0.558 | 7.30E+05 | 0.7436 | 0.2141 | Base to Base |
| 33.3651 | 1.37E+06 | 0.2059 | 5.85E+05 | 0.596 | 0.0803 | Base to Base |
| 34.7807 | 3.81E+06 | 0.5705 | 9.24E+05 | 0.9412 | 0.1338 | Base to Base |
| 35.2633 | 2.05E+06 | 0.3075 | 7.05E+05 | 0.7181 | 0.107 | Base to Base |
| 36.9489 | 5.65E+06 | 0.8458 | 9.78E+05 | 0.9955 | 0.1605 | Base to Base |
| 38.1394 | 2.87E+06 | 0.4303 | 8.43E+05 | 0.8586 | 0.107 | Base to Base |
| 38.6896 | 6.18E+06 | 0.9252 | 1.03E+06 | 1.0486 | 0.2408 | Base to Base |
| 40.5898 | 2.15E+06 | 0.3224 | 5.81E+05 | 0.5917 | 0.1338 | Base to Base |
| 41.5733 | 7.00E+06 | 1.0491 | 9.43E+05 | 0.96 | 0.2408 | Base to Base |
| 42.0674 | 3.46E+06 | 0.5184 | 8.98E+05 | 0.9143 | 0.1338 | Base to Base |
| 42.2718 | 8.14E+06 | 1.2191 | 1.05E+06 | 1.0705 | 0.2676 | Base to Base |
| 42.4327 | 2.55E+06 | 0.3818 | 6.87E+05 | 0.6998 | 0.107 | Base to Base |
| 44.1144 | 7.48E+06 | 1.1211 | 1.08E+06 | 1.1033 | 0.2943 | Base to Base |
| 46.1964 | 6.08E+06 | 0.9103 | 8.94E+05 | 0.9104 | 0.1873 | Base to Base |
| 46.8725 | 3.21E+06 | 0.4811 | 7.64E+05 | 0.7785 | 0.1338 | Base to Base |
| 47.3852 | 1.47E+06 | 0.2207 | 7.06E+05 | 0.7194 | 0.0803 | Base to Base |
| 50.6192 | 1.54E+06 | 0.2307 | 6.74E+05 | 0.6865 | 0.0803 | Base to Base |
| 50.8303 | 3.42E+06 | 0.5122 | 1.03E+06 | 1.0527 | 0.107 | Base to Base |
| 51.2141 | 6.26E+06 | 0.9376 | 1.25E+06 | 1.2757 | 0.1873 | Base to Base |
| 51.531 | 8.74E+06 | 1.3088 | 1.14E+06 | 1.1594 | 0.2408 | Valley |
| 51.6971 | 7.62E+06 | 1.1419 | 1.49E+06 | 1.5193 | 0.1606 | Valley |
| 51.9366 | 3.58E+06 | 0.5358 | 1.13E+06 | 1.146 | 0.1338 | Base to Base |
| 52.5223 | 4.27E+06 | 0.6399 | 1.01E+06 | 1.0277 | 0.1338 | Base to Base |
| 53.2728 | 4.39E+06 | 0.6573 | 7.78E+05 | 0.7923 | 0.1606 | Base to Base |
| 53.8949 | 1.32E+07 | 1.9795 | 1.63E+06 | 1.6568 | 0.3211 | Base to Base |
| 54.4009 | 4.66E+06 | 0.6975 | 1.34E+06 | 1.3633 | 0.107 | Valley |
| 54.5073 | 1.82E+07 | 2.7254 | 1.63E+06 | 1.6624 | 0.2676 | Valley |
| 55.9786 | 4.07E+06 | 0.6101 | 9.34E+05 | 0.9515 | 0.1338 | Base to Base |
| 56.3475 | 1.33E+07 | 1.9928 | 1.13E+06 | 1.1551 | 0.3211 | Valley |
| 56.5847 | 4.61E+06 | 0.6907 | 8.48E+05 | 0.864 | 0.1605 | Valley |
| 56.8275 | 9.26E+06 | 1.3875 | 1.22E+06 | 1.243 | 0.1873 | Valley |
| 57.0145 | 4.88E+06 | 0.7309 | 9.98E+05 | 1.016 | 0.1606 | Valley |
| 57.1188 | 2.45E+06 | 0.3669 | 7.75E+05 | 0.7891 | 0.107 | Base to Base |
| 57.7155 | 6.35E+06 | 0.952 | 1.11E+06 | 1.1327 | 0.1606 | Valley |
| 57.8152 | 9.52E+06 | 1.4268 | 1.38E+06 | 1.4031 | 0.2141 | Valley |

TABLE 21
LC/MS Fingerprint of extract AV016BaSu(65)06(100)
(TIC Spectrum (Q1 +ve mode)

| Time | Area | | Height | | Width | |
|---|---|---|---|---|---|---|
| (min) | (counts) | % Area | (cps) | % Height | (min) | Baseline Type |
| 1.3425 | 1.34E+08 | 15.6624 | 2.35E+07 | 34.2569 | 0.2141 | Base to Base |
| 2.3068 | 6.22E+08 | 72.7416 | 3.24E+07 | 47.2835 | 0.5886 | Base to Base |
| 4.4357 | 5.31E+06 | 0.6201 | 1.63E+06 | 2.3849 | 0.1605 | Base to Base |
| 5.2161 | 2.96E+06 | 0.3463 | 1.29E+06 | 1.8752 | 0.107 | Base to Base |
| 5.6491 | 9.55E+06 | 1.1164 | 1.58E+06 | 2.3015 | 0.1873 | Base to Base |
| 6.6417 | 9.07E+06 | 1.0594 | 2.04E+06 | 2.9753 | 0.1605 | Base to Base |
| 17.6827 | 1.27E+07 | 1.4888 | 1.34E+06 | 1.954 | 0.4281 | Base to Base |
| 54.1984 | 5.00E+07 | 5.8447 | 3.32E+06 | 4.8386 | 0.4548 | Base to Base |
| 57.3586 | 9.59E+06 | 1.1203 | 1.46E+06 | 2.1301 | 0.1873 | Base to Base |

TABLE 22
LC/MS Fingerprint of extract AV016BaSu(105)08(100)
(TIC Spectrum (Q1 +ve mode)

| Time | Area | | Height | | Width | |
|---|---|---|---|---|---|---|
| (min) | (counts) | % Area | (cps) | % Height | (min) | Baseline Type |
| 2.0569 | 2.14E+08 | 27.1727 | 2.15E+07 | 36.3993 | 0.2677 | Base to Base |
| 3.0165 | 5.13E+08 | 65.1587 | 2.56E+07 | 43.3466 | 0.7224 | Base to Base |
| 4.4937 | 1.06E+07 | 1.3463 | 2.07E+06 | 3.4978 | 0.1873 | Base to Base |
| 4.8116 | 9.88E+06 | 1.255 | 1.36E+06 | 2.304 | 0.2141 | Base to Base |
| 7.1128 | 9.55E+06 | 1.2133 | 1.79E+06 | 3.0334 | 0.1606 | Base to Base |
| 20.2582 | 6.97E+06 | 0.8856 | 1.05E+06 | 1.7785 | 0.1873 | Base to Base |
| 21.0285 | 2.37E+06 | 0.3008 | 8.07E+05 | 1.3647 | 0.107 | Base to Base |
| 21.9949 | 5.91E+06 | 0.751 | 1.41E+06 | 2.3921 | 0.1605 | Base to Base |
| 26.1397 | 4.67E+06 | 0.5933 | 1.60E+06 | 2.7139 | 0.1338 | Base to Base |
| 28.479 | 1.04E+07 | 1.3233 | 1.87E+06 | 3.1697 | 0.1873 | Base to Base |

TABLE 23
LC/MS Fingerprint of extract AV016FrDi(65)04(100)
(TIC Spectrum (Q1 +ve mode)

| Time | Area | | Height | | Width | |
|---|---|---|---|---|---|---|
| (min) | (counts) | % Area | (cps) | % Height | (min) | Baseline Type |
| 1.6028 | 1.52E+09 | 29.4877 | 6.12E+07 | 29.3519 | 0.6423 | Valley |
| 2.2961 | 3.50E+09 | 68.0186 | 1.30E+08 | 62.3426 | 0.99 | Valley |
| 4.0708 | 3.33E+06 | 0.0647 | 1.35E+06 | 0.6474 | 0.107 | Base to Base |
| 4.8702 | 1.71E+07 | 0.3327 | 2.48E+06 | 1.1882 | 0.2675 | Base to Base |
| 5.4024 | 9.12E+06 | 0.1773 | 2.48E+06 | 1.1914 | 0.1338 | Base to Base |
| 6.2587 | 4.84E+06 | 0.0941 | 1.53E+06 | 0.7348 | 0.107 | Base to Base |
| 47.0653 | 5.85E+07 | 1.1379 | 2.31E+06 | 1.1097 | 0.6689 | Base to Base |
| 48.8324 | 4.52E+06 | 0.0879 | 1.54E+06 | 0.7369 | 0.1338 | Base to Base |
| 49.5832 | 5.13E+06 | 0.0998 | 1.18E+06 | 0.5668 | 0.1338 | Base to Base |
| 51.2074 | 3.34E+06 | 0.065 | 1.38E+06 | 0.6601 | 0.0802 | Base to Base |
| 51.9303 | 6.03E+06 | 0.1171 | 1.52E+06 | 0.7281 | 0.1338 | Base to Base |
| 57.0683 | 1.63E+07 | 0.3173 | 1.55E+06 | 0.7421 | 0.3211 | Base to Base |

TABLE 24
LC/MS Fingerprint of extract AV016FrSu(105)08(100)
(TIC Spectrum (Q1 +ve mode)
Peak List:

| Time | Area | | Height | | Width | |
|---|---|---|---|---|---|---|
| (min) | (counts) | % Area | (cps) | % Height | (min) | Baseline Type |
| 1.9597 | 2.64E+08 | 29.489 | 2.96E+07 | 45.0514 | 0.2677 | Base to Base |
| 3.1068 | 6.21E+08 | 69.4326 | 3.28E+07 | 49.9315 | 0.6689 | Base to Base |
| 5.6942 | 4.34E+06 | 0.485 | 1.88E+06 | 2.8549 | 0.0802 | Base to Base |
| 22.1023 | 5.31E+06 | 0.5934 | 1.42E+06 | 2.1622 | 0.1605 | Base to Base |

TABLE 25
IC₅₀ values of alpha-glucosidase inhibition activity
of extracts from different T. arjuna plant parts

| | | IC₅₀ |
|---|---|---|
| Plant Part | Extract-ID | (μg/ml) |
| 1. Bark | AV016BaDi(65)04(100) | 13.2 |
| 2. Bark | AV016BaDi(28)04(20) | 14.2 |
| 3. Bark | AV016BaSu(65)01(100)g | 14.3 |
| 4. Bark | AV016BaSu(65)01(100)ng | 15.0 |
| 5. Bark | AV016BaSu(65)04(100) | 16.5 |
| 6. Bark | AV016BaSu(65)06(100) | 24.0 |
| 7. Bark | AV016BaSu(105)08(100) | 31.5 |
| 8. Bark | AV016BaSu(65)09(100) | 42.7 |
| 9. Fruits | AV016FrDi(65)04(100) | 16.3 |
| 10. Fruits | AV016FrSu(105)08(100) | 31.5 |
| 11. Acarbose (Standard) | | 80.7 |

TABLE 26
The effect of insulin and T. arjuna extracts on glucose up-take in 3T3-L1 adipocytes.

| | | cpm | | | | | Stimulation |
|---|---|---|---|---|---|---|---|
| Set | Treatment | Set I | Set II | Set III | Average | SD value | Index |
| 1 | cells alone | 314.6 | 390 | 1200.8 | 635.13 | 491.33 | |
| 2 | cells + Insulin (10 nM) | 2185.8 | 1705.4 | 3010.7 | 2300.63 | 660.18 | 2.6 |
| 3 | cells + Insulin (25 nM) | 4806.8 | 1399.7 | 996.7 | 2401.07 | 2093.15 | 2.8 |
| 4 | cells + Insulin (100 nM) | 2906.5 | 3776.6 | 2215.6 | 2966.23 | 782.21 | 3.7 |
| 5 | cells + AV016BaDi(65)04(100) | 1946.5 | 2570.2 | 3106.7 | 2541.13 | 580.65 | 3.0 |
| | (Ter1) | | | | | | |
| 6 | cells + Insulin (10 nM) + | 2178.5 | 1873 | 1667.3 | 1906.27 | 257.22 | 2.0 |
| | AV016BaDi(65)04(100) | | | | | | |
| | (Ter1) | | | | | | |

Stimulation index = {mean of sample − mean of control (cells alone)/mean of control (cells alone)}

TABLE 27
Anti-obesity potential of Terminalia arjuna extracts.

| | | | Final Reading | |
|---|---|---|---|---|
| | Reading | | (Reading − | |
| Treatment | OD520 nm | Average | Well Blank) | % Inhibition |
| Well Blank (−cells) | 0.887 | | 0 | — |
| Cells Alone | 2.883 | 2.883 | 1.996 | 0.0 |
| | 2.883 | | | |
| Cells + DMSO | 2.786 | 2.786 | 1.899 | 0.0 |
| | 2.786 | | | |
| Cells + AV016BaDi(65)04(100) | 1.428 | 1.344 | 0.457 | 76.3 |
| | 1.26 | | | |
| Cells + AV016BaSu(65)09(100) | 2.105 | 1.616 | 0.729 | 61.9 |
| | 1.127 | | | |
| Cells + AV016BaSu(65)01(100)ng | 1.161 | 1.146 | 0.259 | 86.8 |
| | 1.131 | | | |
| Cells + AV016BaSu(65)01(100)g | 2.23 | 1.6805 | 0.7935 | 58.5 |
| | 1.131 | | | |
| Cells + AV016BaSu(65)04(100) | 1.813 | 1.8535 | 0.9665 | 49.4 |
| | 1.894 | | | |
| Cells + AV016BaSu(65)06(100) | 1.905 | 1.693 | 0.806 | 57.9 |
| | 1.481 | | | |
| Cells + AV016BaDi(28)04(20) | 1.832 | 1.6 | 0.713 | 64.3 |
| | 1.368 | | | |
| Cells + AV016FrDi(65)04(100) | 1.941 | 1.9895 | 1.1025 | 41.9 |
| | 2.038 | | | |
| Cells + +ve Control | 0.774 | 0.807 | −0.08 | 104.0 |
| | 0.84 | | | |

% Inhibition = {(Mean of Control − Mean of Sample)/Mean of Control}*100

TABLE 28
Effect of Terminalia arjuna extracts
on lipid levels in CHO-K1 cells.

| Sr. | | | % Inhibition |
|---|---|---|---|
| No. | Treatment | OD_{520 nm} | w.r.t control |
| 1. | Cells alone + DMSO | 1.206 | 0 |
| 2. | Cells + AV016BaSu(65)09(100) | 0.944 | 22 |
| 3. | Cells + AV016BaSu(65)01(100) | 0.964 | 21 |
| 4. | Cells + AV016BaDi(65)04(100) | 0.844 | 31 |
| 5. | Cells + AV016BaDi(28)04(20) | 0.873 | 28 |

% Inhibition = {(Control − Extract treated/Control) * 100}

TABLE 29
Effect of 20% ethanol extract from T. arjuna bark
AV016BaDi(28)04(20) on HMG-CoA reductase activity.

| | Without Inhibitor | With Inhibitor |
|---|---|---|
| Time (mins) | Reading | Reading |
| 0 | 1.3465 | 1.7372 |
| 1 | 1.3103 | 1.726 |
| 2 | 1.2891 | 1.726 |
| 3 | 1.2768 | 1.726 |
| 4 | 1.2689 | 1.7042 |
| 5 | 1.2534 | 1.7042 |
| Substrate Blank | 0.0277 | 0.0307 |
| | 0 | |
| Final Reading | 0.0654 | 0.0046 |
| % Inhibition | 0 | 93.0 |

% Inhibition = {(Control − Extract treated/Control) * 100}

TABLE 30
Effect of direct 100% ethanol extract from T. arjuna
bark AV016BaDi(65)04(100) on HMG-CoA reductase activity.

| | Without Inhibitor | With Inhibitor |
|---|---|---|
| Time (mins) | Reading | Reading |
| 0 | 1.162 | 1.7815 |
| 1 | 1.1125 | 1.7637 |
| 2 | 1.1048 | 1.7637 |
| 3 | 1.0828 | 1.7637 |
| 4 | 1.0756 | 1.746 |
| 5 | 1.0652 | 1.7467 |
| Substrate Blank | 0.023 | 0.0212 |
| | 0 | |
| Final Reading | 0.0738 | 0.0272 |
| % Inhibition | 0 | 63.1 |

% Inhibition = {(Control − Extract treated/Control) * 100}

TABLE 31
Effect of Terminalia arjuna extracts
on cell viability of HepG2 cells.

| | No. of live | No. of dead |
|---|---|---|
| Treatment | cells | cells |
| Cells | 85 | 20 |
| Cells + DMSO | 82 | 11 |
| Cells + Ascorbic acid | 66.5 | 18.5 |
| Cells + AV016BaSu(65)09(100) | 27 | 7 |
| Cells + AV016BaSu(65)01(100) | 55 | 2 |
| Cells + AV016BaSu(65)04(100) | 11 | 1 |

1. A method for treating a disease selected from the group consisting of obesity, cancer, cardiovascular disease and diabetes in a mammal, wherein said method comprises administering to the mammal an effective non-toxic amount of at least an extract from Terminalia arjuna selected from those as defined in Tables 1-24.

2. The method according to claim 1 wherein the disease is obesity and the extract is selected from the group consisting of AVO1 6BaDi(65)04(100), AVO 16BaDi(28)04(20), AVO 16BaSu(65)09(100), AVO 16BaSu(65)01(100)g, AVO 16BaSu(65)01(100)ng, AVO 16BaSu(65)04(100), AV016BaSu(65)06(100) and AV016FrDi(65)04(100), or a combination of two or more thereof.

3. The method according to claim 1 wherein the disease is cancer and the extract is selected from the group consisting of AV016BaSu(65)09(100), AV016BaSu(65)01(100) and AVO1 6BaSu(65)U4(100), or a combination of two or more thereof.

4. The method according to claim 1 wherein the disease is cardiovascular disease and the extract is selected from the group consisting of AV016BaDi(65)04(100), AV016BaDi(28)04(20), AV016BaSu(65)09(100) and AV016BaSu(65)01(100), or a combination of two or more thereof.

5. The method according to claim 1 wherein the disease is diabetes and the extract is selected from the group consisting of AV016BaDi(65)04(100), AVO 16BaDi(28)04(20), AVO 16BaSu(65)09(100), AVO 16BaSu(65)01(100)g, AVO1 6BaSu(65)01(100)ng, AVOI 6BaSu(65)04(100), AVO 16BaSu(65)06(100), AVO 16BaSu(105)08(100), AVO 16FrDi(65)04(100), and AVO 16FrSu(105)08(100), or a combination of two or more thereof.

6. The method according to claim 1 wherein the disease is diabetes, obesity and cardiovascular disease and the extract is selected from the group consisting of AV016BaDi(65)04(100) and AV016BaDi(28)04(20), or a combination of two.

7. The method according to claim 1 wherein the said treatment is a prophylactic treatment.

8. A pharmaceutical formulation for use in the treatment of a disease selected from the group consisting of diabetes, obesity, cardiovascular disease, and cancer wherein said formulation comprises at least one extract isolated from Terminalia arjuna, and selected from those listed in Tables 1-24, in admixture with a pharmaceutically acceptable carrier.

9-14. (canceled)

15. A method for the preparation of a pharmaceutical formulation comprising bringing into association at least an extract of Terminalia arjuna selected from the group consisting of the extracts having the HPLC and/or MS characteristics shown in Tables 1-24, and a pharmaceutically acceptable carrier therefore.

16. An extract from Terminalia arjuna selected from the group consisting of the extracts having the HPLC and/or MS characteristics shown in Tables 1-24.

17. A comestible comprising at least an extract from Terminalia arjuna selected from the group consisting of the extracts having the HPLC and/or MS characteristics shown in Tables 1-24.

18. The comestible according to claim 17 comprising at least an extract for use in the prophylaxis of cancer.

19. The comestible according to claim 17 comprising at least an extract for use in the prophylaxis of cardiovascular disease.

20. The comestible according to claim 17 comprising at least an extract for use in the prophylaxis of diabetes.

21. The comestible according to claim 17 comprising at least an extract for use in the prophylaxis of obesity.

22. The comestible according to claim 17 comprising at least an extract selected from the group consisting of AV016BaDi(65)04(100) and AVO 16BaDi(28)04(20), either singly or in one or more combinations thereof, for use in the prophylaxis of diabetes, obesity and cardiovascular diseases.

23. A method for the preparation of a medicament for the treatment of disease selected from the group consisting of diabetes, cancer, cardiovascular disease and obesity, wherein said method comprises the use of an extract selected from the group consisting of the extracts having the HPLC and/or MS characteristics shown in Tables 1-24.

24. The method according to claim 23 wherein said method utilizes an extract selected from the group consisting of AVO 16BaDi(65)04(100), AVO 16BaDi(28)04(20), AVO 16BaSu(65)09(100), AVO 16BaSu(65)01(100)g, AVO 16BaSu(65)01(100)ng, AVO 16BaSu(65)04(100), AVO 16BaSu(65)06(100), AV016BaSu(105)08(100), AV016FrDi(65)04(100) and AV016FrSu(105)08(100) for the preparation of a medicament for the treatment or prophylaxis of diabetes and/or related diseases.

25. The method according to claim 23 wherein said method utilizes an extract selected from the group consisting AV016BaDi(65)04(100), AV016BaDi(28)04(20), AV016BaSu(65)09(100), AV016BaSu(65)01(100)g, AV016BaSu(65)01(100)ng, AV016BaSu(65)04(100) AV016BaSu(65)06(100) and AV016FrDi(65)04(100) for the preparation of a medicament for the treatment or prophylaxis of obesity.

26. The method according to claim 23 wherein said method utilizes an extract selected from the group consisting of AV016BaDi(65)04(100), AV016BaDi(28)04(20), AV016BaSu(65)09(100) and AV016BaSu(65)01(100) either singly or in one or more combinations thereof for the preparation of a medicament for the treatment or prophylaxis of cardiovascular disease.

27. The method according to claim 23 wherein said method utilizes an extract selected from the group consisting of AV016BaSu(65)09(100), AV016BaSu(65)01(100) and AV016BaSu(65)04(100) either singly or in one or more combinations thereof for the preparation of a medicament for the treatment or prophylaxis of cancer.

28. The method according to claim 23 wherein said method utilizes an extract AV016BaDi(65)04(100) and AV016BaDi(28)04(20) either singly or in one or more combinations thereof for the preparation of a medicament for the treatment or prophylaxis of diabetes, obesity, and cardiovascular diseases.
